(19) United States
(12) Patent Application Publication
(10) Pub. No.: US 2007/0154999 A1
(43) Pub. Date: Jul. 5, 2007

(54) SUCCINIC ACID - PRODUCING BACTERIUM AND PROCESS FOR PRODUCING SUCCINIC ACID

(75) Inventors: Keita FUKUI, Kanagawa (JP); Jun NAKAMURA, Kanagawa (JP); Kayo AKIYAMA, Kanagawa (JP); Hiroyuki KOJIMA, Kanagawa (JP)

Correspondence Address: GREENBLUM & BERNSTEIN, P.L.C., 1950 ROLAND CLARKE PLACE, RESTON, VA, 20191, US

(73) Assignees: AJINOMOTO CO., INC.,, Tokyo (JP); MITSUBISHI CHEMICAL CORPORATION, Tokyo (JP)

(21) Appl. No.: 11/560,937

(22) Filed: Nov. 17, 2006

Related U.S. Application Data
(63) Continuation-in-part of application No. PCT/JP05/09233, filed on May 20, 2005.

(30) Foreign Application Priority Data
May 20, 2004 (JP) 2004-150672

(51) Int. Cl. C12P 7/46; C12N 15/74; C12N 1/21

(52) U.S. Cl. 435/145; 435/252.3; 435/471

(57) ABSTRACT

Succinic acid is produced by using a coryneform bacterium which has succinic acid-producing ability and has been modified so that an activity of pyruvate oxidase is decreased.

### TECHNICAL FIELD

The present invention relates to a fermentation industry, and to a process for efficiently producing succinic acid by a fermentation method using a coryneform bacterium.

### BACKGROUND ART

For production of non-amino organic acids including succinic acid by fermentation, usually, anaerobic bacteria such as those belonging to the genus Anaerobiospirillum or Actinobacillus are used (Patent Document 1 or 2, and Non-Patent Document 1). The use of anaerobic bacteria makes the yield of products high, while such bacteria require many nutrients for proliferation and therefore, there is a need for adding a large amount of organic nitrogen sources such as corn steep liquor (CSL) to a medium. The addition of abundant amounts of organic nitrogen sources not only leads to an increase in cost of the medium but also leads to an increase in cost of purification for isolating the product, which is uneconomical.

Furthermore, a method, which comprises culturing aerobic bacteria such as coryneform bacteria under an aerobic condition to proliferate bacterial cells and then collecting and washing the cells to use them as resting bacteria to produce succinic acid without oxygen aeration, has been known (Patent Document 3 and 4). This method is economical because the bacterial cells can grow sufficiently in a simple medium, into which a small amount of organic nitrogen is added for proliferation of bacterial cells, but this method is still to be improved in terms of the amount of generated succinic acid, the concentration thereof, and the production rate thereof per bacterial cells as well as simplification of production process, and the like.

Furthermore, when aerobic bacteria such as coryneform bacteria are cultured under oxygen-limited conditions, organic acids other than a desired substance such as lactic acid and acetic acid are excessively accumulated as by-products, resulting in suppressed growth of bacterial cells and significantly decreased productivity in fermentation. In addition, excessive amounts of counterions to neutralize the organic acids generated as by-products are required, thereby resulting in being uneconomical. To solve such problems, reduction in lactate generated as a by-product has been performed by using a coryneform bacterium having a reduced lactate dehydrogenase activity (Patent Document 5).

### BACKGROUND ART

However, even if the above-mentioned coryneform bacterium having decreased lactate dehydrogenase activity is used, a large amount of acetic acid is generated as a by-product. As means for solving the problem of reducing acetic acid in a culture medium, there have been known a method of enhancing expression of an acetic acid assimilating gene (aceP) in a bacterium belonging to the genus Escherichia (Patent Document 6), a method of enhancing expression of a gene encoding ACE protein in a bacterium belonging to the genus Escherichia (Patent Document 7), and the like. Those methods are intended to reduce generation of acetic acid as a by-product by actively assimilating acetic acid released into a culture medium. Meanwhile, as methods of suppressing generation of acetic acid as a by-product by suppressing biosynthesis of acetic acid in a bacterium belonging to the genus Escherichia, there is known a method of producing succinic acid using Escherichia coli in which phosphoacetyltransferase and lactate dehydrogenase are deficient (patent Document 8).

As enzymes responsible for assimilation of acetic acid in a coryneform bacterium, there have been reported acetate kinase and phosphotransacetylase (Non-Patent Document 2). On the other hand, it is assumed that not only the above-mentioned enzymes but also a plurality of enzymes including pyruvate oxidase (Patent Document 9), acylphosphatase, aldehyde dehydrogenase and acetyl-CoA hydrolase are responsible for generation of acetic acid, but a specific enzyme that contributes to synthesis of acetic acid has not been clarified. Therefore, there has not been known a method of producing succinic acid using a strain of a coryneform bacterium having decreased acetic acid biosynthetic enzyme activity.

Pyruvate oxidase is an enzyme which produces acetic acid from pyruvic acid and water (EC 1.2.2.2), and there have been known a method of producing an L-amino acid using enterobacteria in which pyruvate oxidase is deficient (Patent Document 10), a method of producing D-pantothenic acid using enterobacteria in which pyruvate oxidase is deficient (Patent Document 11), and a method of producing D-pantothenic acid using a coryneform bacterium in which pyruvate oxidase is deficient (Patent Document 12).

A gene sequence of pyruvate oxidase of Corynebacterium glutamicum has been identified, and there has been known a method of producing an L-amino acid using a coryneform bacterium which is modified so that the expression of a pyruvate oxidase gene is decreased (Patent Document 13). However, contribution of pyruvate oxidase to succinic acid-biosynthetic system in a coryneform bacterium has been unknown, and no report has been provided on expression analysis of pyruvate oxidase gene under anaerobic conditions.

Patent Document 1: U.S. Pat. No. 5,143,833

Patent Document 2: U.S. Pat. No. 5,504,004

Patent Document 3: JP11-113588A

Patent Document 4: JP11-196888A

Patent Document 5: JP11-206385A

Patent Document 6: JP06-14781A

Patent Document 7: JP07-67683A

Patent Document 8: WO 99/06532

Patent Document 9: EP 1096013A

Patent Document 10: WO 02/36797

Patent Document 11: WO 02/072855

Patent Document 12: WO 02/29020

Patent Document 13: EP1108790A

Non-Patent Document 1: International Journal of Systematic Bacteriology, vol. 49, p 207-216, 1999

Non-Patent Document 2: Microbiology, 1999, February; 145 (Pt2): 503-13

### DISCLOSURE OF THE INVENTION

An object of the present invention is to provide a coryneform bacterium capable of efficiently producing succinic acid.

The inventors of the present invention have intensively studied to solve the aforementioned problems, and as a result, they found that generation of acetic acid as a by-product is reduced and a succinic acid is efficiently produced in a coryneform bacterium by decreasing a pyruvate oxidase activity, thereby accomplished the present invention.

That is, the present invention is as follows.
(1) A coryneform bacterium having a succinic acid-producing ability, wherein said bacterium has been modified so that an activity of pyruvate oxidase is decreased.
(2) The coryneform bacterium according to (1), wherein the pyruvate oxidase is a protein as described in the following (A) or (B):

(A) a protein having an amino acid sequence of SEQ ID NO: 49; or

(B) a protein having an amino acid sequence of SEQ ID NO: 49 including substitution, deletion, insertion, or addition of one or several amino acids, and having a pyruvate oxidase activity.
(3) The coryneform bacterium according to (1) or (2), wherein the pyruvate oxidase activity is decreased by disruption of a pyruvate oxidase gene on a chromosome.
(4) The coryneform bacterium according to (3), wherein the pyruvate oxidase gene is a DNA as described in the following (a) or (b);

(a) a DNA comprising the nucleotide sequence of nucleotide numbers 996-2732 in SEQ ID NO: 48; or

(b) a DNA that hybridizes with the nucleotide sequence of nucleotide numbers 996-2732 in SEQ ID NO: 48 or a probe that can be prepared from the nucleotide sequence under stringent conditions, and encodes a protein having a pyruvate oxidase activity.
(5) The coryneform bacterium according to any one of (1) to (4), wherein said bacterium has been further modified so that activities of one or more of phosphotransacetylase, acetate kinase and acetyl-CoA Hydrolase is decreased.
(6) The coryneform bacterium according to any one of (1) to (5), wherein said bacterium has been further modified so that an activity of lactate dehydrogenase is decreased.
(7) The coryneform bacterium according to any one of (1) to (6), wherein said bacterium has been further modified so that an activity of pyruvate carboxylase is increased.
(8) A method for producing succinic acid, comprising

allowing the coryneform bacterium according to any one of (1) to (7) or a treated product thereof to act on an organic raw material in a reaction liquid containing carbonate ion, bicarbonate ion or carbon dioxide to produce and accumulate succinic acid in the reaction liquid; and

collecting succinic acid from the reaction liquid.
(9) The production method according to (8), wherein the bacterium or treated product thereof is allowed to act on the organic raw material under anaerobic conditions.
(10) A method for producing a succinic acid-containing polymer, comprising the steps of producing succinic acid by the method according to (8) or (9) and polymerizing the obtained succinic acid.

### BRIEF DESCRIPTION OF THE DRAWINGS

FIG. 1 shows the procedures for constructing plasmid pBS3.

FIG. 2 shows the procedures for construction plasmid pBS4S.

FIG. 2 shows the procedures for construction plasmid pΔldh56-L

FIG. 2 shows the procedures for construction plasmid pBS4S::Δpox B.

FIG. 5 is a graph showing ratio of acetic acid as a byproduct in the poxB-disrupted strain and the control strain.

FIG. 6 shows the procedures for construction plasmid pBS5T::Δack.

FIG. 7 shows the procedures for construction plasmid pBS5T::Δpta-ack.

FIG. 8 shows the procedures for construction plasmid pBS5T::Δpox B.

FIG. 9 shows the procedures for construction plasmid pBS4S::Δach.

FIG. 10 shows the procedures for construction plasmid pBS5T::Δacp.

FIG. 11 shows the procedures for construction plasmid pBS5T.

### DESCRIPTION OF PREFERRED EMBODIMENTS

Hereinafter, embodiments of the present invention will be described in detail.

<1> Coryneform Bacterium to be used in the Present Invention

In the present invention the term “coryneform bacterium” includes a bacterium which had been classified as the genus Brevibacterium but now classified as the genus Corynebacterium (Int. J. Syst. Bacteriol., 41, 255 (1981)), and it also includes a bacterium belonging to the genus Brevibacterium, which is very closely related to Corynebacterium. Examples of such coryneform bacteria include the followings.

Corynebacterium acetoacidophilum

Corynebacterium acetoglutamicum

Corynebacterium alkanolyticum

Corynebacterium callunae

Corynebacterium glutamicum

Corynebacterium lilium

Corynebacterium melassecola

Corynebacterium thermoaminogenes

Corynebacterium herculis

Brevibacterium divaricatum

Brevibacterium flavum

Brevibacterium immariophilum

Brevibacterium lactofermentum

Brevibacterium roseum

Brevibacterium saccharolyticum

Brevibacterium thiogenitalis

Corynebacterium ammoniagenes

Brevibacterium album

Brevibacterium selinum

Microbacterium ammoniaphilum

In the present invention, the term “succinic acid-producing ability” means an ability to accumulate succinic acid in a medium when the coryneform bacterium of the present invention is cultured. The succinic acid-producing ability may be a feature inherent to a coryneform bacterium or a feature provided by breeding.

To provide the succinic acid-producing ability by breeding, there may be applied methods that have been employed in breeding of coryneform bacteria, which include acquisition of metabolic regulation mutant strains, creation of a recombinant strain having an enhanced biosynthetic enzyme for a desired substance, and the like (Amino Acid Fermentation, Japan Scientific Societies Press, the first edition published on May 30, 1986, p 77-100). In these methods, one or tow or three or more features such as metabolic regulation mutations and enhancement of biosynthetic enzymes for a desired substance may be provided. Imparting properties such as metabolic regulation mutations and enhancement of biosynthetic enzymes may be combined. An example of succinic acid-producing enzyme includes pyruvate carboxylase as described below.

Particularly preferably specific examples of a coryneform bacteria having a succinic acid-producing ability include Brevibacterium flavum MJ233Δldh strain having decreased lactate dehydrogenase activity (JP11-206385A), Brevibacterium flavum MJ233/pPCPYC strain having enhanced activity of pyruvate carboxylase or phosphoenol pyruvate carboxylase (WO 01/027258 and JP11-196887A), Brevibacterium flavum MJ-233 (FERM PB-1497), Brevibacterium flavum MJ-233 AB-41 (FERM BP-1498), Brevibacterium ammoniagenes ATCC6872, Corynebacterium glutamicum ATCC31831, and Brevibacterium lactofermentum ATCC13869. Since Brevibacterium flavum may be currently classified as Corynebacterium glutamicum (Lielbl, W., Ehrmann, J., Ludwig, W. and Schleifer, K. H., International Journal of Systematic Bacteriology, 1991, vol. 41, p 255-260), the aforementioned Brevibacterium flavum MJ-233 strain and its mutant MJ-233 AB-41 strain, are defined as the same strains as Corynebacterium glutamicum MJ-233 strain and Corynebacterium glutamicum MJ-233 AB-41 strain, respectively.

<2> Construction of the Coryneform bacterium of the Present Invention

The coryneform bacterium of the present invention is a coryneform bacterium that has the above-mentioned succinic acid-producing ability and modified so that pyruvate oxidase activity is decreased.

In breeding of the coryneform bacterium of the present invention, there is no preference as to which of the provision of a succinic acid-producing ability and the modification for decreasing pyruvate oxidase (EC 3.1.2.1) activity is performed first.

The term “pyruvate oxidase activity” refers to an activity to catalyze a reaction to generate acetic acid from pyruvic acid and water. The phrase “modified so that pyruvate oxidase activity is decreased” means that pyruvate oxidase activity is decreased as compared to a specific activity of an unmodified strain, for example, a wild-type coryneform bacterium. The pyruvate oxidase activity is preferably decreased to 50% or less per bacterial cell, more preferably 30% or less, further more preferably 10% or less per bacterial cell as compared to an unmodified strain. Herein, examples of a wild-type coryneform bacterium to be used as a control include Brevibacterium lactofermentum ATCC13869 (wild-type strain) and Brevibacterium lactofermentum Δldh strain (unmodified strain). The pyruvate oxidase activity can be determined according to the method of Chang Y. et al. (Chang Y. and Cronan J. E. JR, J. Bacteriol. 151, 1279-1289 (1982)).

Examples of pyruvate oxidase having the above-mentioned activity include a protein having an amino acid sequence of SEQ ID NO: 49. In addition, as long as the protein has a pyruvate oxidase activity, it may be a protein having an amino acid sequence of SEQ ID NO: 49 including substitution, deletion, insertion, or addition of one or several amino acids. Here, for example, the term “several” means 2 to 20, preferably 2 to 10, or more preferably 2 to 5.

The phrase “modified so that pyruvate oxidase activity is decreased” includes decrease in the number of molecules of pyruvate oxidase per cell, decrease in the pyruvate oxidase activity per molecule and the like. Specifically, it is achieved by disrupting a gene encoding pyruvate oxidase on a chromosome, modification of an expression regulatory sequence such as promoter, Shine-Dalgamo (SD) sequence, or the like. Examples of the pyruvate oxidase gene on a chromosome, modification of an expression regulatory sequence such as promoter, Shine-Dalgamo (SD) sequence, or the like. Examples of the pyruvate oxidase gene on a chromosome include a DNA having the nucleotide sequence of nucleotide numbers 996-2732 in SEQ ID NO: 48. Also, it may be a DNA that hybridizes with a nucleotide sequence of nucleotide numbers 996-2732 in SEQ ID NO: 48 or a probe that can be prepared from the nucleotide sequence under stringent conditions as long as it encodes a protein having the pyruvate oxidase activity. The term “stringent conditions” refers to conditions under which a so-called specific hybrid is formed and non-specific hybrid is not formed. It is difficult to clearly define the conditions by numeric value, but examples thereof include conditions that comprises washing once, preferably twice or three times at salt concentrations corresponding to 1×SSC, 0.1% SDS, preferably 0.1×SSC, 0.1% SDS at 60° C.

The pyruvate oxidase gene (hereinafter, referred to as poxB gene) can be obtained by, for example, cloning performed by synthesizing synthetic oligonucleotides based on a sequence of Corynebacterium glutamicum registered in GenBank (a complementary strand of 2776766-2778505 of GenBank Accession No. NC_{—}003450), and performing PCR using a chromosome of Corynebacterium glutamicum as a template. In addition, there may also be used a sequence of a coryneform bacterium such as Brevibacterium lactofermentum having a nucleotide sequence determined by the recent genome project. Chromosomal DNA can be prepared from a bacterium as a DNA donor by, for example, the method of Saito and Miura (H. Saito and k. Miura, Biochem. Biophys. Acta, 72, 619 (1963), Experimental Manual for Biotechnology, edited by The Society for Biotechnology, Japan, p 97-98, Baifukan Co., Ltd., 1992) or the like.

The poxB gene thus prepared or a part thereof can be used for gene disruption. A gene to be used for gene disruption only needs to have homology enough to cause homologous recombination with a poxB gene to be disrupted on a chromosome of a coryneform bacterium (e.g. a gene having the nucleotide sequence of nucleotide numbers 996-2732 in SEQ ID NO: 48), so such a homologous gene may be used. Here, the homology enough to cause homologous recombination is preferably not less than 70%, more preferably not less than 80%, further more preferably not less than 90%, particularly preferably not less than 95%. Further, DNAs capable of hybridizing with the above-mentioned gene under stringent conditions can cause homologous recombination. The term “stringent conditions” refers to conditions under which a so-called specific hybrid is formed and non-specific hybrid is not formed. It is difficult to clearly define the conditions by numeric value, but examples thereof include, conditions that comprises washing once, preferably twice or three times at salt concentrations corresponding to 1×SSC, 0.1% SDS, preferably 0.1×SSC, 0.1% SDS at 60° C.

For example, by using the above-mentioned gene, a deleted-form of poxB gene, which is modified so as not to produce pyruvate oxidase that normally functions by deleting a partial sequence of the poxB gene, is prepared, and a coryneform bacterium is transformed with a DNA including the gene to cause recombination between the deleted-form gene and a gene on a chromosome, to thereby disrupt the poxB gene on a chromosome. Such a gene disruption by gene substitution using homologous recombination has already been established, and examples thereof include a method using a linear DNA and a method using a plasmid containing a temperature-sensitive replication origin (U.S. Pat. No. 6,303,383 or JP05-007491A). Further, the above-mentioned gene disruption by gene substitution using homologous recombination may also be performed using a plasmid having no replication ability in a host.

For example, a poxB gene on a chromosome of a host can be substituted by a deleted-form of poxB gene in accordance with the following procedures. First, a plasmid for recombination is prepared by inserting a temperature-sensitive replication origin, deleted-form of poxB gene, sacB gene encoding levansucrase and marker gene resistant to a drug such as chloramphenicol.

Here, sacB gene encoding levansucrase is a gene which is used for efficiently selecting a strain in which a vector portion has been excised from a chromosome (Schafer, A. et al., Gene 145 (1994) 69-73). That is, when levansucrase is expressed in a coryneform bacterium, levan generated by assimilation of sucrose acts lethally on the bacterium, so the bacterium cannot grow. Therefore, if a bacterial strain in which a vector carrying levansucrase remains on a chromosome is cultured on a sucrose-containing plate, it cannot grow. As a result, only a bacterial strain from which the vector has been excised can be selected on the sucrose-containing plate.

Genes each having the following sequences can be used as a sacB gene or homologous gene thereof.

Bacillus subtilis: sacB GenBank Accession Number X02730 (SEQ ID NO: 41)

Bacillus amyloliquefaciens: sacB GenBank Accession Number X52988

Zymomonas mobilis: sacB GenBank Accession Number L33402

Bacillus stearothermophilus: surB GenBank Accession Number U34874

Lactobacillus sanfranciscensis: frfA GenBank Accession Number AJ508391

Acetobacter xylinus: lsxA GenBank Accession Number AB034152

Gluconacetobacter diazotrophicus: lsdA GenBank Accession Number L41732

A coryneform bacterium is transformed with the above-mentioned recombinant plasmid. The transformation can be performed in accordance with a transformation method which has been previously reported. Examples of the method include, a method of increasing permeability of a DNA by treating cells of a recipient bacterium with calcium chloride as reported for Escherichia coli K-12 (Mandel, M. and Higa, A., J. Mol. Biol., 53, 159 (1970) and, a method of preparing competent cells using proliferating cells for introduction of DNA as reported for Bacillus subtilis (Duncan, C. H., Wilson, G. A and Young, F. E, Gene 1, 53 (1977)). Alternatively, as reported for Bacillus subtilis, actinomycetes and yeasts, a method of introducing a recombinant DNA into cells of a DNA recipient bacterium (Chang, S. and Choen, S. N., Molec. Gen. Genet., 168, 111 (1979); Bibb, M. J., Ward, J. M., and Hopwood, O. A., Nature, 274, 398 (1978); Hinnen, A., Hicks, J. B. and Fink, G. R., Proc. Natl. Acad. Sci. USA, 75 1928 (1978)) may also be applied. In addition, a coryneform bacterium may be transformed by the electric pulse method (Sugimoto et al., JP02-207791A).

Examples of a temperature-sensitive plasmid for a coryneform bacterium include p48K and pSFKT2 (JP2000-262288A), and pHSC4 (France Patent No. 2667875 (1992) and JP05-7491A). These plasmids are autonomously replicable in a coryneform bacterium at least at 25° C., but they are not autonomously replicable at 37° C. Escherichia coli AJ12571 having pHSC4 has been deposited with an Accession no. FERM P-11763 at National Institute of Bioscience and Human-Technology, Agency of industrial Science and Technology, Ministry of International Trade and Industry (currently, International Patent Organism Depositary, National Institute of Advanced Industrial Science and Technology) (Central 6, 1-1-1 Higashi, Tsukuba, Ibaraki, 305-54566 Japan) on Oct. 11, 1990, and then transferred to an international deposit under the provisions of Budapest Treaty on Aug. 26, 1991 with an Accession No. FERM BP-3524.

A transformant obtained as described above is cultured at a temperature at which the temperature-sensitive replication origin does not function (25° C.), to thereby obtain a strain into which the plasmid has been introduced. The plasmid-introduced strain is cultured at high temperature to excise the temperature-sensitive plasmid, and the bacterial strain is applied onto a plate containing an antibiotic. The temperature-sensitive plasmid cannot replicate at high temperature. Therefore, a bacterial strain from which the plasmid has been excised cannot grow on a plate containing an antibiotic, but a bacterial strain in which recombination has occurred between the poxB gene on the plasmid and the poxB gene on a chromosome appears at a very low frequency.

In the strain obtained by introducing the recombinant DNA into a chromosome as described above, recombination occurs with the poxB gene sequence that is originally present on a chromosome, and two fusion genes of the chromosomal poxB gene and the deleted-form of poxB gene are inserted into a chromosome so that other portions of the recombinant DNA (vector part, temperature-sensitive replication origin and drug-resistance marker) are present between the fusion genes.

Then, in order to leave only the deleted-form of poxB gene on a chromosomal DNA, the gene is eliminated together with the vector portion (the temperature-sensitive replication origin and drug-resistance marker) from the chromosomal. This procedure causes a case where the normal poxB gene remains on the chromosomal DNA and the deleted-form of poxB gene is excised, or to the contrary, a case where the normal poxB gene is excised and the deleted-form of poxB gene remains on chromosomal DNA. In both cases, when culture is performed at a temperature that allows a temperature-sensitive replication origin to function, the cleaved DNA is kept in a cell as a plasmid. Next, when culture is performed at a temperature that does not allow a temperature-sensitive replication origin to function, the poxB gene on the plasmid is eliminated from the cell together with the plasmid. Then, a strain in which the deleted-form of poxB gene remains on the chromosome, is selected by PCR, Southern hybridization, or the like, to thereby yield a strain in which the poxB gene is disrupted.

In the case where a plasmid having no replicability in a coryneform bacterium is used instead of the above-mentioned temperature-sensitive plasmid, gene disruption can also be performed in a similar way. The plasmid having no replicability in a coryneform bacterium is preferably a plasmid having a replicability in Escherichia coli, and examples thereof include pHSG299 (Takara Bio Inc.) and pHSG399 (Takara Bio Inc.).

Meanwhile, examples of a method of decreasing an activity of pyruvate oxidase include not only the above-mentioned genetic engineering method but also a method comprising treating a coryneform bacterium with ultraviolet irradiation or with a mutagenesis agent to be generally used for mutation such as N-methyl-N′-nitro-N-nitrosoguanidine (NTG) and nitrous acid, and selecting a bacterial strain having decreased pyruvate oxidase activity.

In the present invention, it is more effective to use a bacterial strain modified so that a lactate dehydrogenase (hereinafter, referred to as LDH) activity is decreased in addition to the above-mentioned pyruvate oxidase activity. The lactate dehydrogenase activity means an activity to catalyze a reaction to generate lactic acid by reducing pyruvic acid using NADH as a coenzyme. The phrase “lactate dehydrogenase activity is decreased” means the LDH activity is decreased as compared to an LDH-unmodified strain. The LDH activity is decreased than an LDH-unmodified strain or a wild-type strain, and it is preferably decreased to 50% or less, more preferably 30% or less, particularly preferably 10% or less per bacterial cell. LDII activity may also be completely eliminated. The decreased LDH activity can be confirmed by determining the LDH activity by the method of L. Kanarek et al. (L. Kanarek and R. L. Hill, J. Biol. Chem. 239, 4202 (1964)). The coryneform bacterium of the present invention can be obtained by preparing a coryneform bacterium having decreased LDH activity and modifying it so that the pyruvate oxidase activity is decreased. However, there is no preference in the order for performing the modification to decrease the LDH activity and the modification to decrease the pyruvate oxidase activity. As an ldh gene, there may be used, for example, a gene having the sequence of SEQ ID NO: 43, and gene disruption may be performed in a similar manner as in the case of the above-mentioned poxB gene.

In the present invention, it is more effective to use a bacterial strain modified so that activity of any one of phosphotransacetylase (hereinafter, referred to as PTA), acetate kinase (hereinafter, referred to as ACK), acetyl-CoA hydrolase (ACH) is decreased, in addition to the decrease in pyruvate oxidase activity. It is further effective to use a bacterial strain modified so that activities of two or more of the enzymes are decreased, and is particularly effective to use a bacterial strain modified so that all of the activities are decreased. It is further effective to use a bacterial strain further deficient in acylphosphatase.

In the present invention, the phosphotransacetylase (PTA) activity means an activity to catalyze a reaction to generate acetyl phosphate by transferring phosphate to acetyl-CoA. The acetate kinase (ACK) activity means an activity to catalyze a reaction to generate acetic acid from acetyl phosphate and ADP. The acetyl-CoA hydrolase (ACH) activity means an activity to catalyze a reaction to generate acetic acid from acetyl-CoA and water. The acylphosphatase (ACP) activity means an activity to catalyze a reaction to generate phosphoric acid and acetic acid, or carboxylic acid and phosphoric acid from acetyl phosphate.

Decreasing these activities can be performed by disrupting genes encoding the above-mentioned enzymes, or by modifying expression regulatory sequences such as promoter and Shine-Dalgamo (SD) sequence of genes encoding the enzymes. The disruption of a gene can be performed in the same way as the above-mentioned method of disrupting poxB gene.

As genes encoding the enzymes, there may be used, for example, the following genes of Corynebacterium glutamicum registered in GenBank:

pta (phosphoacetyltransferase) gene: NCgl2657 of GenBank Accession No. NC_{—}003450 (a complementary strand of nucleotide numbers 2936506-2937891 of NC_{—}003450) (the nucleotide numbers 956-1942 in SEQ ID NO: 45)

ack (acetate kinase) gene: NCgl2656 of GenBank Accession No. NC_{—}003450 (a complementary strand of nucleotide numbers 2935313-2936506 of NC_{—}003450) (the nucleotide numbers 1945-3135 in SEQ ID NO: 45).

ach (acetyl-CoA hydrolase) gene: NCgl2480 of GenBank Accession No. NC_{—}003450 (a complementary strand of nucleotide number 2729376-2730884 of NC_{—}003450) (SEQ ID NO: 50)

acp (acylphosphatase gene: NCgl1987 of GENEBANK accession No. NC_{—}003450 (a complementary strand of nucleotide number 2183107-2183391 of NC_{—}003450) (SEQ ID NO: 52)

The phrase “phosphotransacetylase (hereinafter, referred to as PTA) activity is decreased” means that PTA activity is decreased as compared to PTA-unmodified strain. The PTA activity is lower than PTA-unmodified strain or a wild-type strain, and it is preferably decreased to 50% or less, more preferably 30% or less, particularly preferably 10% or less per bacterial cell. PTA activity may also be completely eliminated. The decreased PTA activity can be confirmed by determining the PTA activity by the method of Klotzsch et al. (Klotzsch H. R., Meth Enzymol. 12, 381-386 (1969)). A coryneform bacterium having decreased activities of POXB and PTA can be obtained by constructing a coryneform bacterium having decreased POXB activity and modifying it so that the PTA activity is decreased. However, there is no preference in the order for performing the modification to decrease PTA activity and the modification to decrease POXB activity.

The phrase “acetate kinase (hereinafter, referred to as ACK) activity is decreased” means that ACK activity is decreased as compared to a wild-type strain or ACK-unmodified strain. The ACK activity is lower than an ACK-unmodified strain or a wild-type strain, and it is preferably decreased to 50% or less, more preferably 30% or less, particularly preferably 10% or less per bacterial cell as compared to an ACK-unmodified strain. ACK activity may also be completely eliminated. The decreased ACK activity can be confirmed by determining the ACK activity by the method of Ramponi et al. (Ramponi G, Meth. Enzymol. 42, 409-426 (1975)). A coryneform bacterium having decreased activities of POXB and ACK can be obtained by constructing a coryneform bacterium having decreased POXB activity and modifying it so that the ACK activity is decreased. However, there is no preference in the order for performing the modification to decrease ACK activity and the modification to decrease POXB activity.

The phrase “acetyl-CoA (hereinafter, referred to as ACH) activity is decreased” means that the activity is lower than an ACH-unmodified strain or a wild-type strain, and the activity is preferably decreased to 50% or less, more preferably 30% or less, particularly preferably 10% or less per bacterial cell as compared to an ACH-unmodified strain. ACH activity may also be completely eliminated. The decreased ACH activity can be confirmed by determining the ACH activity by the method of Gergely, J. et al. (Gergely, J., Hele, P. & Ramkrishnan, C. V. (1952) J. Biol. Chem. 198 p. 323-334). A coryneform bacterium having decreased activities of ACH and POXB can be obtained by constructing a coryneform bacterium having decreased ACH activity and modifying it so that the POXB activity is decreased. However, there is no preference between the modification to decrease POXB activity and the modification to decrease ACH activity.

The phrase “acylphosphatase (hereinafter, referred to as ACP) activity is decreased” means that ACP activity is decreased as compared to a wild-type strain or an ACP-unmodified strain. The ACP activity is lower than a wild-type strain or an ACP-unmodified strain, and it is preferably decreased to 50% or less per bacterial cell, more desirably 10% or less per bacterial cell as compared to an ACP-unmodified strain. ACP activity may also be completely eliminated. The decreased ACP activity can be confirmed by determining the ACP activity by the same method as for the ACK activity (Ramponi G., Meth. Enzymol. 42, 409-426 (1975)). A coryneform bacterium having decreased activities of POXB and ACP can be obtained by constructing a coryneform bacterium having decreased POXB activity and modifying it so that the ACP activity is decreased. However, there is no preference in the order for performing the modification to decrease ACP activity and the modification to decrease POXB activity.

Meanwhile, in the present invention, there may be used a bacterium modified so that an activity of pyruvate carboxylase (hereinafter, referred to as PC) is increased in addition to the decrease in POXB activity. The phrase “pyruvate carboxylase activity is increased” means that PC activity is increased as compared to a wild-type strain or an unmodified strain such as a parent strain. The PC activity can be determined by the method of Peters-Wendisch P. G et al. (Peters-Wendisch P. G. et al. Microbiology 143, 1095-1103 (1997)).

As a PC gene encoding a PC protein to be used in the method of the present invention, there may be employed a gene whose nucleotide sequence has been determined, or a gene obtained by isolating a DNA fragment that encodes a protein having PC activity from a chromosome of microorganisms, animals, plants, or the like according to the method described below and determining its nucleotide sequence. Further, after determination of the nucleotide sequence, a gene synthesized based on the sequence may be used. For example, there may be used a pyruvate carboxylase gene of Corynebacterium glutamicum ATCC13032 (GenBank Accession no. NCgl0659 gene: SEQ ID NO: 60). Further, there may also be used PC genes derived from the following organisms.

Human [Biochem. Biophys. Res. Comm., 202, 1009-1014, (1994)]

Mouse [Proc. Natl. Acad. Sci. USA., 90, 1766-1779 (1993)]

Rate [GENE, 165, 331-332, (1995)]

Yeast; Saccharomyces cerevisiae [Mol. Gen. Genet., 229, 307-315, (1991)]
Schizosaccharomyces pombe [DDBJ Accession No.: D78170]

Bacillus stearothermophilus [GENE, 191, 47-50, (1997)]

Rhizobium etli [J. Bacteriol., 178, 5960-5970, (1996)]

A DNA fragment containing a PC gene can be expressed by inserting the DNA fragment into a suitable expression plasmid such as pUC118 (Takara Bio Inc.), and introducing into a suitable host microorganism such as Escherichia coli JM109 (Takara Bio Inc.). The expressed PC gene product, which is pyruvate carboxylase, can be confirmed by determining PC activity by the known method as described above in the transformant, and then comparing the determined PC activity with PC activity with PC activity of a crude enzyme solution extracted from a non-transformant strain. The DAN fragment containing PC gene is inserted into a suitable plasmid such as a plasmid vector containing at least a gene responsible for replication function of the plasmid in coryneform bacteria, thereby a recombinant plasmid capable of highly expressing PC in coryneform bacteria can be obtained. Here, in the recombinant plasmid, a promoter for expression of PC gene may be a promoter of coryneform bacteria. However, it is not limited to such a promoter, and any promoter can be used as long as it has a nucleotide sequence capable of initiating transcription of PC gene.

Plasmid vectors, into which PC gene can be introduced, are not limited as long as they contain at least a gene responsible for replication function in coryneform bacteria. Specific examples thereof include: plasmid pCRY30 described in JP03-210184A; plasmids pCRY21, pCRY2KE, pCRY2KX, pCRY31, pCRY3KE, and pCRY3KX each described in JP02-72876A and U.S. Pat. No. 5,185,262; plasmids pCRY2 and pCRY3 each described in JP01-191686A; pAM330 described in JP58-67679A; pHM1519 described in JP58-77895A; pAJ655, pAJA611, and pAJ1844 each described in JP58-192900A; pCG1 described in JP57-134500A; pCG2 described in JP58-35197A; and pCGG4 and pCG11 each described in JP57-183799A.

Of those, plasmid vectors used in host-vector system for coryneform bacteria are preferably those having a gene responsible for replication function of the plasmid in coryneform bacteria and a gene responsible for stabilization function of the plasmid in coryneform bacteria. For instance, pCRY30, pCRY21, pCRY2KE, pCRY2KX, pCRY31, pCRY3KE, and pCRY3KX can be preferably used.

Coryneform bacteria having enhanced PC gene expression can be obtained by transforming a coryneform bacterium, for example, Brevibacterium lactofermentum 2256 strain (ATCC13869) with a recombinant vector prepared by inserting PC gene into an appropriate site of a plasmid vector which is replicable in aerobic coryneform bacteria as described above. Transformation can be carried out by, for example, the electric pulse method (Res. Microbiol., Vol. 144, p. 181-185, 1993). PC activity can also be increased by enhancing gene expression by introduction, substitution, amplification or the like of PC gene on a chromosome by a known homologous recombination method. By disrupting the poxB gene in a strain which highly expresses the PC gene, a bacterial strain with enhanced PC activity and decreased pyruvate oxidase activity can be obtained. There is no preference in the order for performing modifications to decrease pyruvate oxidase activity and to enhance PC activity.

Moreover, in the present invention, a bacterium, which has been modified so that activities of pyruvate oxidase, ACH, PTA and ACK are decreased and further modified so that LDH activity is decreased and PC activity is increased, is particularly effectively used for production of a substance, especially for production of succinic acid.

<3> Production of Succinic Acid using the Bacterium of the Present Invention

Succinic acid can be efficiently produced by culturing the thus obtained coryneform bacterium in a medium to produce and accumulate succinic acid in the medium and collecting succinic acid from the medium.

Upon use of the above-mentioned bacterium in reaction for producing succinic acid, the bacterium subjected to slant culture on such a solid medium as an agar medium may be used directly for the reaction, but a bacterium obtained by culturing the above-mentioned bacterium in a liquid medium (seed culture) in advance may be preferably used. Succinic acid may be produced by allowing the seed-cultured bacterium to react with an organic material while the bacterium is proliferating in a medium containing the organic raw material. In addition, succinic acid can also be produced by harvesting bacterial cells which has been proliferated and then reacting the bacterial cells with an organic raw material in reaction liquid containing the organic raw material. Further, for the purpose of using an aerobic coryneform bacterium in the method of the present invention, it is preferable to use the aerobic coryneform bacterium for the reaction after culturing the bacterium under a normal aerobic condition. The medium to be used for culture may be any medium normally used for culturing microorganisms. For instance, conventional media, which can be prepared by adding natural nutrient sources such as neat extract, yeast extract and peptone to a composition made of inorganic salts such as ammonium sulfate, potassium phosphate and magnesium sulfate, can be sued. In the case of harvesting and using the bacterial cells after culture, the bacterial cells are harvested by centrifugation, membrane separation, or the like, and then used for the reaction.

In the present invention, a treated product of bacterial cells can also be used. For instance, the treated products of bacterial cells include immobilized bacterial cells which are immobilized on acrylamide, carrageenan or the like, disrupted bacterial cells, centrifugal supernatant thereof, or fractions obtained by partially purifying the supernatant with an ammonium sulfate treatment or the like.

An organic raw material to be sued for the production method of the present invention is not particularly limited as long as it is a carbon source which can be assimilated by the microorganism described herein to produce succinic acid. In general, there is used a fermentable carbohydrate including: a carbohydrate such as galactose, lactose, glucose, fructose, glycerol, sucrose, saccharose, starch and cellulose; polyalcohol such as glycerin, mannitol, xylitol and ribitol. Of those, glucose, fructose and glycerol are preferable, and glucose is particularly preferable.

In addition, a saccharified starch liquid, molasses and the like, which contain any one of the above-mentioned fermentable carbohydrates, can also be used. Any one of those fermentable carbonhydrates may be used alone or may be used in combination. The concentration at which the above-mentioned organic raw material is used is not particularly limited, but it is advantageous to increase the concentration as high as possible within the range that does not inhibit the production of succinic acid. The reaction is generally performed under the presence of the organic raw material in the range of 5 to 30% (w/v), preferably 10 to 20% (w/v). The organic raw material may be additionally added according to a decrease in the above-mentioned organic raw material when the reaction progresses.

The reaction liquid containing the organic raw material is not particularly limited. The reaction liquid to be used may be water, buffer, medium or the like, but the medium is most preferable. The reaction liquid is preferably one containing a nitrogen source, inorganic salts and the like. Here, the nitrogen source is not particularly limited as long as it can be assimilated by the microorganism described herein to produce succinic acid. Specific examples of the nitrogen source include various organic and inorganic nitrogen compounds such as ammonium salts, nitrate, urea, soybean hydrolysate, casein hydrolysate, peptone, yeast extract, meat extract, and corn steep liquor. Examples of the inorganic salts include various kinds of phosphoric acid salts, sulfuric acid salts and metal salts of magnesium, potassium, manganese, iron, zinc, and the like. In addition, components that promote growth of bacterial cells including: vitamins such as biotin, pantothenic acid, inositol and nicotinic acid; nucleotides; and amino acids, may be added if necessary. Further, it is preferable that an appropriate amount of a commercially available antifoaming agent is added to the reaction liquid to suppress foaming at the time of reaction.

pH of the reaction liquid can be adjusted by adding sodium carbonate, sodium bicarbonate, potassium carbonate, potassium bicarbonate, magnesium carbonate, sodium hydroxide, calcium hydroxide, magnesium hydroxide, or the like. The pH for the reaction is usually 5 to 10, preferably 6 to 9.5, so the pH of the reaction liquid is adjusted within the above-mentioned range with an alkaline material, carbonate, urea, or the like during the reaction, if necessary.

The medium preferably contains carbonate ion, bicarbonate ion or carbonic acid gas (carbon dioxide). The carbonate ion or bicarbonate ion is supplied from magnesium carbonate, sodium carbonate, sodium bicarbonate, potassium carbonate, or potassium bicarbonate, which can also be used as a neutralizing agent. However, if necessary, the carbonate ion or bicarbonate ion can also be supplied from carbonic acid or bicarbonic acid, or salts thereof, or carbonic acid gas. Specific examples of the salts of carbonic acid or bicarbonic acid include magnesium carbonate, ammonium carbonate, sodium carbonate, potassium carbonate, ammonium bicarbonate, sodium bicarbonate, and potassium bicarbonate. In addition, the carbonate ion or bicarbonate ion is added at a concentration of 0.001 to 5 M, preferably 0.1 to 3 M, and more preferably 1 to 2 M. When carbonic acid gas is contained, the amount of the carbonic acid gas to be contained is 50 mg to 25 g, preferably 100 mg to 15 g, and more preferably 150 mg to 10 g per liter of the liquid.

The optimal temperature at which the bacterium to be used in the reaction grow is generally in the range of 25° C. to 35° C. On the other hand, the temperature at the time of reaction is generally in the range of 25° C. to 40° C., preferably in the range of 30° C. to 37° C. The amount of bacterial cells to be used in the reaction is not particularly limited, but the amount is adjusted in the range of 1 to 700 g/L, preferably 10 to 500 g/L, and more preferably 20 to 400 g/L. The time period of the reaction is preferably 1 to 168 hours, more preferably 3 to 72 hours.

Upon culture of the bacterium, it is necessary to supply oxygen by aeration and agitation. On the other hand, the reaction for producing succinic acid may be performed with aeration and agitation, or may be performed under an anaerobic condition with neither aeration nor supply of oxygen. Here, the term “anaerobic condition” means that the reaction is conducted while keeping the dissolved oxygen low in the liquid. In this case, it is preferable to carry out the reaction at a dissolved oxygen of 0 to 2 ppm, preferably 0 to 1 ppm, and more preferably 0 to 0.5 ppm. For that purpose, there may be used a method in which a vessel is hermetically sealed to carry out the reaction without aeration; a method in which an inert gas such as a nitrogen gas is supplied to carry out the reaction; a method in which aeration with an inert gas containing carbonic acid gas is performed; and the like.

Succinic acid accumulated in the reaction liquid (culture solution) can be isolated and purified from the reaction liquid according to a conventional procedure. To be specific, succinic acid can be isolated and purified by removing solid materials including bacterial cells through centrifugation, filtration or the like, and desalting the solution with an ion exchange resin or the like, followed by crystallization or column chromatography from the solution.

In the present invention, after production of succinic acid by the method of the present invention as described above, a polymerization reaction is carried out using the obtained succinic acid as a raw material to produce a succinic acid-containing polymer. The succinic acid-containing polymer may be a homopolymer or a copolymer with other polymer raw materials. In recent years, environment-friendly industrial products are on the increase, and polymers prepared by using raw materials of a plant origin have been attracting attention. The succinic acid to be produced in the present invention can be processed into polymers such as polyester and polyamide and then used. Specific examples of the succinic acid-containing polymer include a succinic acid-containing polyester obtained through polymerization between a diol such as butanediol or ethylene glycol and succinic acid, and a succinic acid-containing polyamide obtained through polymerization between a diamine such as hexamethylenediamine and succinic acid.

Further, succinic acid or a composition containing succinic acid which can be obtained by the production method of the present invention can be used as food additives, pharmaceuticals, cosmetics, and the like.

### EXAMPLES

Hereinafter, the present invention will be described in further detail with reference to examples.

### Example 1

### <1> Construction of a Disruption Vector Carrying sacB Gene

(A) Construction of pBS3

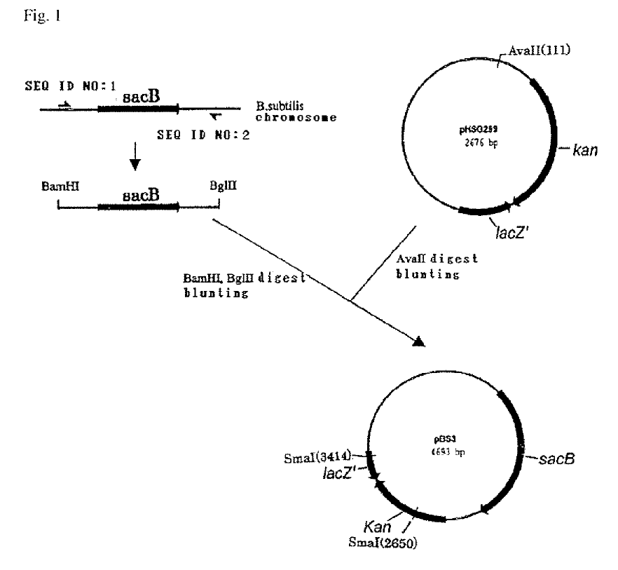

The sacB gene was obtained by PCR using a chromosomal DNA of Bacillus subtilis as a template and SEQ ID NOS: 1 and 2 as primers. PCR was carried out using LA Taq (Takara Bio Inc.) in such a way that one cycle of heat-retention at 94° C. for 5 minutes was performed and then a cycle of denaturation at 94° C. for 30 seconds, annealing at 49° C. for 30 seconds and elongation at 72° C. for 2 minutes was repeated 25 times. The PCR product thus obtained was purified by a conventional procedure and then digested with BglII and BamHI, followed by blunt-ending. The fragment was inserted into a site of pHSG299 which had been digested with AvaII and blunt-ended. Competent cells of Escherichia coli JM109 (Takara Bio Inc.) were used for transformation with this DNA and the transformed cells were applied on an LB medium containing 25 μg/ml kanamycin (hereinafter, abbreviated as Km), followed by overnight culture. Subsequently, appeared colonies were picked up, and single colonies were isolated, thereby transformants were obtained. Plasmids were extracted from transformants, and a plasmid into which a PCR product of interest was inserted was named pBS3. FIG. 1 shows the construction procedures of pBS3.

(B) Construction of pBS4S

SmaI site in the kanamycin resistance gene sequence present on pBS3 was disrupted by crossover PCR-mediated nucleotide substitution causing no amino acid substitution to obtain a plasmid. First, PCR was carried out using a pBS3 as a template and synthetic DNAs of SEQ ID NOS: 3 and 4 as primers, thereby amplified product of N-terminal region of the kanamycin resistance gene was obtained. On the other hand, to obtain amplified product of C-terminal region of the Km resistance gene, PCR was carried out using pBS3 as a template and synthetic DNAs of SEQ ID NOS: 5 and 6 as primers. The PCR was carried out using Pyrobest DNA Polymerase (Takara Bio Inc.) in such a way that one cycle of heat-retention at 98° C. for 5 minutes was performed and then a cycle of denaturation at 98° C. for 10 seconds, annealing at 57° C. for 30 seconds and elongation at 72° C. for 1 minute was repeated 25 times, to thereby yield a PCR product of interest. SEQ ID NOS: 4 and 5 are partially complementary to each other, and the SmaI site in the sequence is disrupted by nucleotide substitution causing no amino acid substitution. Next, to obtain a fragment of a mutant kanamycin resistance gene in which the SmaI site is disrupted, the gene products of the N-terminal and C-terminal regions of the above-mentioned kanamycin resistance gene were mixed at an approximately equimolar concentration, and PCR was carried out using the mixture of the gene products as templates and synthetic DNAs of SEQ ID NOS: 3 and 6 as primers, to thereby yield amplified product of a mutation-introduced Km resistance gene. PCR was carried out using Pyrobest DNA Polymerase (Takara Bio Inc.) in such a way that one cycle of heat-retention at 98° C. for 5 minutes is performed and then a cycle of denaturation at 98° C. for 10 seconds, annealing at 57° C. for 30 seconds and elongation at 72° C. for 1.5 minutes was repeated 25 times, to thereby yield a PCR product of interest.

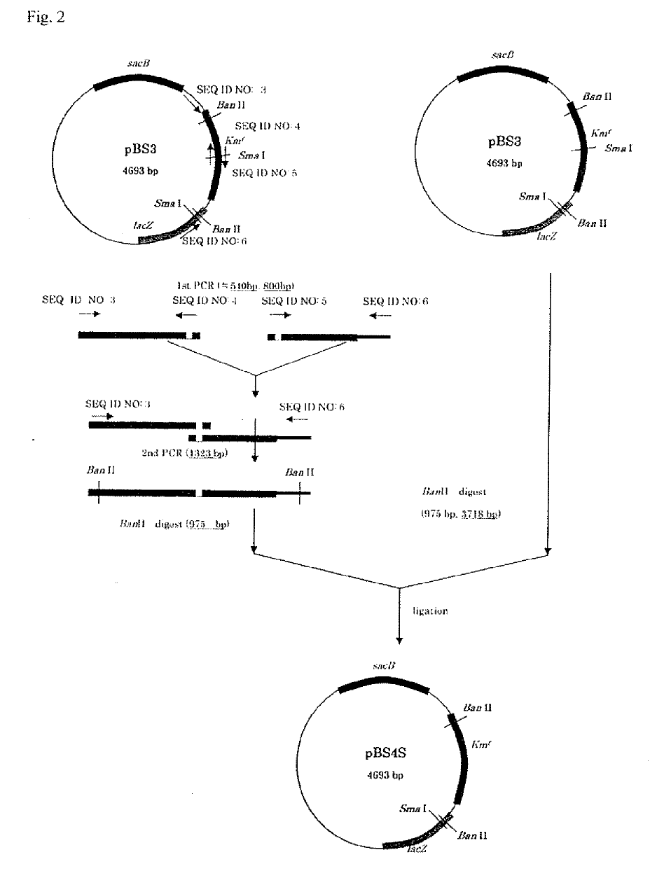

The PCR product was purified by a conventional procedure and then digested with BanII, followed by insertion into BanII site of the above-mentioned pBS3. Competent cells of Escherichia coli JM109 (Takara Bio Inc.) were used for transformation with this DNA and transformed cells were applied on an LB medium containing 25 μg/ml of kanamycin, followed by overnight culture. Subsequently, appeared colonies were picked up, and single colonies were isolated, thereby transformants were obtained. Plasmids were extracted from the transformants, and a plasmid into which a PCR product of interest was inserted was named pBS4S. FIG. 2 shows the construction procedures of pBS4S.

(C) Construction of pBS5T

A plasmid was constructed by inserting a temperature-sensitive replication origin for a coryneform bacterium into pBS4S constructed in the above-mentioned (B). That is, a temperature-sensitive replication origin for a coryneform bacterium was obtained by digesting pHSC4 (JP05-7491A) with BamHI and SmaI, followed by blunt-ending, and the temperature-sensitive replication origin was inserted into a blunt-ended NdeI site of pBS4S. Competent cells of Escherichia coli JM109 (Takara Bio Inc.) were used for transformation with this DNA and transformed cells were applied on an LB medium containing 25 μg/ml of Km, followed by overnight culture. Subsequently, appeared colonies were picked up, and single colonies were isolated, thereby transformants were obtained. Plasmids were extracted from the transformants, and a plasmid into which a PCR product of interest was inserted was named pBS5T. FIG. 11 shows the construction procedures of pBS5T.

### Example 2

### Construction of LDH Gene-Disrupted Strain

(A) Cloning a Fragment for Disrupting Lactate Dehydrogenase Gene

A frament of a lactate dehydrogenase gene (hereinafter, abbreviated as ldh gene) derived from Brevibacterium lactofermentum 2256 strain in which ORF thereof was deleted was obtained by crossover PCR using as primers synthetic DNAs designed based on the nucleotide sequence (Ncg12810 of GenBank Database Accession No. NC_{—}003450) of the gene of Corynebacterium glutamicum ATCC13032 (GenBank Database Accession No. NC_{—}003450), which has already been disclosed. That is, PCR was carried out by a conventional procedure using a chromosomal DNA of Brevibacterium lactofermentum 2256 strain as a template and synthetic DNAs of SEQ ID NOS: 7 and 8 as primers, thereby amplified product of the N-terminal region of the ldh gene was obtained. On the other hand, to obtain amplified product of the C-terminal region of the ldh gene, PCR was carried out by a conventional procedure using a genomic DNA of Brevibacterium lactofermentum 2256 as a template and synthetic DNAs of SEQ ID NOS: 9 and 10 as primers. SEQ ID NOS: 8 and 9 are complementary to each other and have structures for deleting the entire sequences of ldh ORF.

Brevibacterium lactofermentum 2256 strain is available from the American Type Culture Collection (ATCC) (Address: ATCC, P.O. Box 1549, Manassas, Va. 20108 United States of America).

Next, to obtain a fragment of the ldh gene in which its internal sequence is deleted, the above-mentioned gene products of the N-terminal and C-terminal regions of ldh were mixed at an approximately equimolar concentration, and PCR was carried out by a conventional procedure using the mixture of the gene products as templates and synthetic DNAs of SEQ ID NOS: 11 and 12 as primers, to thereby yield amplified product of the mutation-introduced ldh gene. The PCR product thus obtained was purified by a conventional procedure and then digested with SalI, followed by insertion into SalI site of the above-mentioned pBS4S. Competent cells of Escherichia coli JM109 (Takara Bio Inc.) were used for transformation with this DNA and transformed cells were applied on an LB medium containing 100 μM of IPTG, 40 μg/ml of X-Gal, and 25 μg/ml of Km, followed by overnight culture. Subsequently, appeared white colonies were picked up, and single colonies were isolated, thereby transformants were obtained. Plasmids were extracted from the transformants, and a plasmid into which a PCR product of interest was inserted was named pΔlhd56-1. FIG. 3 shows the construction procedures of the plasmid.

(B) Preparation of ldh-disrupted Strain

the pΔldh56-1 obtained by the above-mentioned (A) does not contain a region that enables autonomous replication in a cell of a coryneform bacterium. Therefore, when a coryneform bacterium is transformed with this plasmid, a strain in which the plasmid is integrated into a chromosome by homologous recombination appears at a very low frequency as a transformant. Brevibacterium lactofermentum 2256 strain was transformed using a high concentration of the plasmid pΔlhd56-1 by the electric pulse method, and the transformed cells were applied on CM-Dex medium (5 g/L of glucose, 10 g/L of polypeptone, 10 g/L of yeast extract, 1 g/L of KH₂PO₄, 0.4 g/L of MgSO₄.7H₂O, 0.01 g/L of FeSO₄.7H₂O, 0.01 g/L of MnSO₄.7H₂O, 3 g/L of urea, 1.2 g/L of soybean hydrolysate, pH 7.5 (KOH)) containing 25 μg/ml of kanamycin, followed by culture at 31.5° C. for about 30 hours. A strain grown on the medium contains the kanamycin resistance gene and sacB gene which are derived from the plasmid on the genome, as a result of homologous recombination between the ldh gene fragment on the plasmid and the ldh gene on a genome of Brevibacterium lactofermentum 2256 strain.

Next, the single cross-over recombinant was cultured at 31.5° C. overnight in CM-Dex liquid medium not containing kanamycin, and after suitable dilution, it was applied on 10% sucrose-containing Dex-S10 medium (100 g/L of sucrose, 10 g/L of polypeptone, 10 g/L of yeast extract, 1 g/L of KH₂PO₄, 0.4 g/L of MgSO₄.7H₂O, 0.01 g/L of FeSO₄.7H₂O, 0.01 g/L of MnSO₄.4H₂O, 3 g/L of urea, 1.2 g/L of soybean hydrolysate, 10 μg/L of biotin, pH 7.5 (KOH) not containing kanamycin, followed by culture at 31.5° C. for about 30 hours. As a result, about 50 strains, which were considered to become sucrose-insensitive due to elimination of the sacB gene by second homologous recombination, were obtained.

The strains thus obtained include: a strain in which ldh gene was replaced by the mutant type derived from pΔldh56-1; and a strain in which ldh gene reverted to the wild type. Whether the ldh gene is the mutant type or the wild type can be confirmed easily by directly subjecting the bacterial strains obtained by culturing on Dex-S10 agar medium to PCR and detecting their ldh gene. In PCR analysis using primers (SEQ ID NOS: 7 and 10) for amplifying ldh gene, a strain which yielded a PCR product having a smaller size than that of a product obtained by PCR using a chromosomal DNA of the 2256 strain as a template was defined as an ldh-disrupted strain and used in the following experiments. As a result of the analysis of the sucrose-insensitive strains by the above-mentioned method, a strain carrying only the mutant type gene was selected and named 2256Δ(ldh) strain. Also, the strain was used as a parent strain for modification in the following examples.

### Example 3

### Construction of Pyruvate Oxidase Gene-Disrupted Strain

(A) Cloning of a Fragment for Disrupting Pyruvate Oxidase Gene

A fragment of a pyruvate oxidase gene (hereinafter, abbreviated as poxB gene) derived from Brevibacterium lactofermentum 2256 strain in which ORF thereof was deleted was obtained by crossover PCR using as primers synthetic DNAs designed based on the nucleotide sequence of the gene of Corynebacterium glutamicum ATCC 13032 (NCgl252I of GenBank Database Accession no. NC_{—}003450), which has already been disclosed. That is, PCR was carried out by a conventional procedure using a genomic DNA of Brevibacterium lactofermentum 2256 strain as a template and synthetic DNAs of SEQ ID NOS: 29 and 30 as primers, thereby amplified product of N-terminal region of the poxB gene was obtained.

On the other hand, to obtain an amplified product of C-terminal region of the poxB gene, PCR was carried out using a genomic DNA of Brevibacterium lactofermentum 2256 strain as a template and synthetic DNAs of SEQ ID NOS: 31 and 32 as primers. SEQ ID NOS: 30 and 31 are complementary to each other. PCR was carried out using KOD-plus-(TOYOBO) in such a way that one cycle of heat-retention at 94° C. for 2 minutes was performed and then a cycle of denaturation at 94° C. for 10 seconds, annealing at 55° C. for 30 seconds and elongation at 68° C. for 40 seconds was repeated 30 times, for the N-terminal and C-terminal regions. Next, to obtain a fragment of a poxB gene in which its internal sequence is deleted, the above-described amplified products of the N-terminal and C-terminal regions of poxB were mixed at an approximately equimolar concentration, and PCR was carried out using the mixture as templates and synthetic DNAs of SEQ ID NOS. 33 and 34 as primers, to thereby yield an amplified product of a mutation-introduced poxB gene. The PCR was carried out using KOD-plus-(TOYOBO) in such a way that one cycle of heat-retention at 94° C. for 2 minutes was performed and then a cycle of denaturation at 94° C. for 10 seconds, annealing at 55° C. for 30 seconds and elongation at 68° C. for 70 seconds was repeated 30 times, to thereby yield an amplified product of the mutation-introduced poxB gene of interest.

The PCR product thus obtained was purified by a conventional procedure and then digested with XbaI, followed by insertion into XbaI site of the pBS4S constructed in the above-mentioned Example 1(B). Competent cells of Escherichia coli (JM109 (Takara Bio Inc.) were used for transformation with this DNA and transformed cells were applied on an LB medium containing 100 μM of IPTG, 40 μg/ml of X-Gal, and 25 μg/ml of kanamaycin, followed by overnight culture. Subsequently, appeared white colonies were picked up, and single colonies were isolated, thereby transformants were obtained. Plasmids were extracted from the transformants, and a plasmid into which a PCR product of interest was inserted was named pBS4S::ΔpoxB. FIG. 4 shows the construction procedures of the pBS4S::ΔpoxB.

(B) Preparation of poxB-disrupted Strain

The pBS4S::ΔpoxB obtained in the above-mentioned (A) does not contain a region that enables autonomous replication in a cell of a coryneform bacterium. Therefore, when a coryneform bacterium is transformed with the plasmid, the strain in which the plasmid is integrated into a chromosome by homologous recombination appears at a very low frequency as a transformant. Brevibacterium lactofermentum 2256Δ(ldh) strain prepared in Example 2 was transformed using a high concentration of the plasmid pBS4S::ΔpoxB by the electric pulse method, and then applied on CM-Dex medium containing 25 μg/ml of kanamycin, followed by culture at 31.5° C. for about 30 hours. The strain grown on the medium contains a kanamycin resistance gene and a sacB gene which are derived from the plasmid on the genome, as a result of homologous recombination between the poxB gene fragment on the plasmid and the poxB gene on a genome of Brevibacterium lactofermentum 2256Δ(ldh) strain.

next, the single crossover recombinant was cultured at 31.5° C. overnight in CM-Dex liquid medium not containing kanamycin, and then, after suitable dilution, it was applied on 10% sucrose-containing Dex-S10 medium not containing kanamycin, followed by culture at 31.5° C. for about 30 hours.

As a result, about 30 strains, which were considered to become sucrose-insensitive due to elimination of the sacB gene by the second homologous recombination, were obtained.

The thus obtained strains include: a strain in which poxB gene was replaced by the mutant type derived from pBS4S::ΔpoxB; and a strain in which poxB gene reverted to the wild type. Whether the poxB gene is the mutant type or the wild type can be confirmed easily by directly subjecting a bacterial strain obtained through culture on a Dex-S10 agar medium to PCR and detecting the poxB gene. Analysis of the poxB gene by using primers (SEQ ID NOS: 29 and 32) for PCR amplification should result in a DNA fragment of 2.4 kb for the wild type and a DNA fragment of 1.2 kb for the mutant type having the deleted region. As a result of the analysis of the sucrose-insensitive strain by the above-mentioned method, a strain carrying only the mutant type gene was selected and named 2256Δ(ldh, poxB). The strain is also called a poxB-disrupted strain, herein.

### Example 4

### Succinic Acid Production of the poxB-disrupted Strain

(A) Evaluation of Culture of the poxB-disrupted Strain

Brevibacterium lactofermentum 2256Δ(ldh) strain and 2256Δ(ldh, poxB) strain were used for culture for producing succinic acid as described below. The bacterial cells of the 2256Δ(ldh) strain and 2256Δ(ldh, poxB) strain obtained by culturing them on a CM-Dex plate medium were inoculated into 3 ml of a seed medium (20 g/L of glucose, 4 g/L of Urea, 14 g/L of (NH₄)₂SO₄, 0.5 g/L of KH₂PO₄, 0.5 g/L of K₂HPO₄, 0.5 g/L of MgSO₄.7H₂O, 0.02 g/L of FeSO₄.7H₂O, 0.02 g/L of MnSO₄.7H₂O, 200 μg/L of biotin, 200 μg/L of VB₁.HCl, 1 g/L of yeast extract, and 1 g/L of casamino acid; with no pH adjustment; glucose was added after being independently sterilized). Shaking culture was performed in a test tube at 31.5° C. for about 16 hours under an aerobic condition.

After that, 3 ml of a main medium A (100 g/L of glucose, 15 g/L of sodium sulfite, and 71.4 g/L of MgCO₃ was added into the tube. For preventing aeration, the succinic acid production culture was carried out while the tube was sealed hermetically with a silicon cap. The culture was performed by shaking at 31.5° C. for about 48 hours and terminated before sugar in the medium was exhausted.

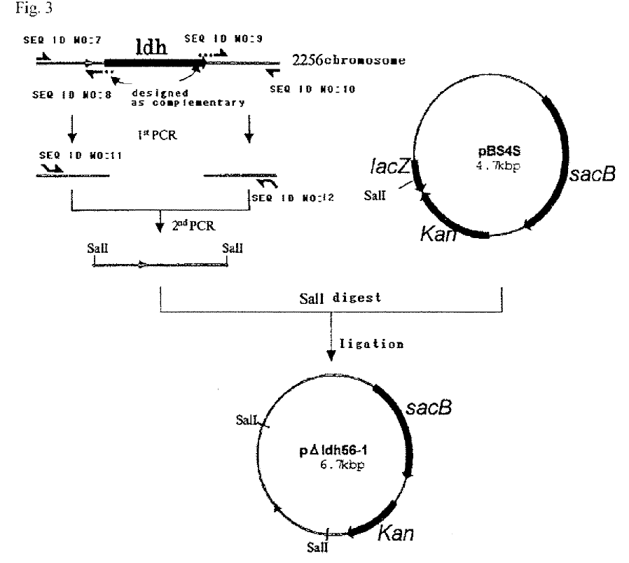

After completion of the culture, the accumulation amounts of succinic acid and by-product acetic acid in the medium were analyzed by liquid chromatography after the medium had been suitably diluted. A column obtained by connecting two pieces of Shim-pack SCR-102H (Shimadzu) in series was used, and the sample was eluted at 40° C. by using 5 mM p-toluene sulfonic acid. The eluent was neutralized by using 20 mM Bis-Tris aqueous solution containing 5 mM p-toluene sulfonic acid and 100 μM of EDTA. The succinic acid and acetic acid were each measured by determining the electric conductivity by means of CDD-10AD (Shimadzu). The obtained results are shown in Table 1 and FIG. 5.

In the case of 2256Δ(ldh, poxB) strain, the succinic acid production was equal to the parent strain 2256Δ(ldh), but ratio of acetic acid with respect to succinic acid was about one third to two third of the 2256Δ(ldh, poxB) strain. These results indicated that eliminating or decreasing poxB activity is effective for reducing acetic acid under anaerobic conditions.

TABLE 1
Production of succinic acid, acetic acid and pyruvic acid
by the poxB-disrupted strain

| | Succinic | Acetic | Pyruvic | Pyruvic acid/ | Acetic acid/ |
|---|---|---|---|---|---|
| | acid | acid | acid | succinic acid | succinic acid |
| | (g/L) | (g/L) | (g/L) | (%) | (%) |
| 2256Δldh | 38.5 | 3.6 | 8.2 | 21.4 | 9.4 |
| | 45.1 | 3.6 | 8.2 | 18.3 | 7.9 |
| | 42.2 | 3.5 | 8.5 | 20.1 | 8.2 |
| 2256Δ | 42.0 | 2.3 | 9.6 | 22.8 | 5.4 |
| (ldh, poxB) | 42.4 | 2.2 | 9.5 | 22.4 | 5.2 |
| | 35.7 | 2.6 | 9.1 | 25.5 | 7.3 |
| | 38.4 | 2.3 | 9.6 | 25.1 | 6.1 |
| | 40.9 | 2.3 | 9.7 | 23.7 | 5.7 |
| | 49.1 | 1.9 | 9.7 | 19.8 | 3.8 |
| | 51.3 | 1.5 | 8.8 | 17.2 | 2.9 |
| | 48.3 | 1.6 | 9.2 | 19.0 | 3.4 |
| | 51.2 | 1.9 | 8.8 | 17.2 | 3.6 |
| | 38.4 | 2.2 | 9.7 | 25.2 | 5.8 |
| | 43.8 | 2.4 | 9.5 | 21.8 | 5.5 |
| | 37.9 | 2.2 | 9.4 | 24.9 | 5.9 |

### Example 5

### Construction of poxB, pta, ack-disrupted Strain and poxB, pta, ack, ach-disrupted Strain

(5-1) <Construction of Acetate Kinase Gene-Disrupted Strain>

(A) Cloning of a Fragment for Disrupting Acetate Kinase Gene

A fragment of an acetate kinase gene (hereinafter, abbreviated as ack) of Brevibacterium lactofermentum 2256 strain in which ORF thereof was deleted was obtained by crossover PCR using as primers synthetic DNAs designed based on the nucleotide sequence of the gene of Corynebacterium glutamicum ATCC13032 (NCgl 2656 of GenBank Database Accession No. NC_{—}003450; SEQ ID NO:45), which has already been disclosed. That is, PCR was carried out using a genomic DNA of Brevibacterium lactofermentum 2256 strain as a template and synthetic DNAs of SEQ ID NOS: 13 and 14 as primers, thereby amplified product of N-terminal region of the ack gene was obtained. On the other hand, to obtain amplified product of C-terminal region of the ack gene, PCR was carried out using a genomic DNA of Brevibacterium lactofermentum 2256 strain as a template and synthetic DNAs of SEQ ID NOS: 15 and 16 as primers. SEQ ID NOS: 14 and 15 are partially complementary to each other. The PCR was carried out using KOD-plus-(TOYOBO) in such a way that one cycle of heat-retention at 94° C. for 2 minutes was performed and then, for the N-terminal region, a cycle of denaturing at 94° C. for 10 seconds, annealing at 55° C. for 30 seconds and elongation at 68° C. for 30 seconds, and for the C-terminal region, a cycle of denaturation at 94° C. for 10 seconds, annealing at 55° C. for 30 seconds and elongation at 68° C. for 2 minutes were repeated 30 times, respectively. Next, to obtain a fragment of ack gene in which its internal sequence is deleted, the gene products of the above-mentioned N-terminal and C-terminal regions of ack were mixed at an approximately equimolar concentration, and PCR was carried out using the mixture of the gene products as templates and synthetic DNAs of SEQ ID NOS: 17 and 18 as primers, to thereby yield amplified product of a mutation-introduced ack gene. The PCR was carried out using KOD-plus-(TOYOBO) in such a way that one cycle of heat-retention at 94° C. for 2 minutes was performed and then a cycle of denaturation at 94° C. for 10 seconds, annealing at 55° for 30 seconds and elongation at 68° C. for 2.5 minutes was repeated 30 times, to thereby yield an amplified product of the mutation-introduced ack gene of interest.

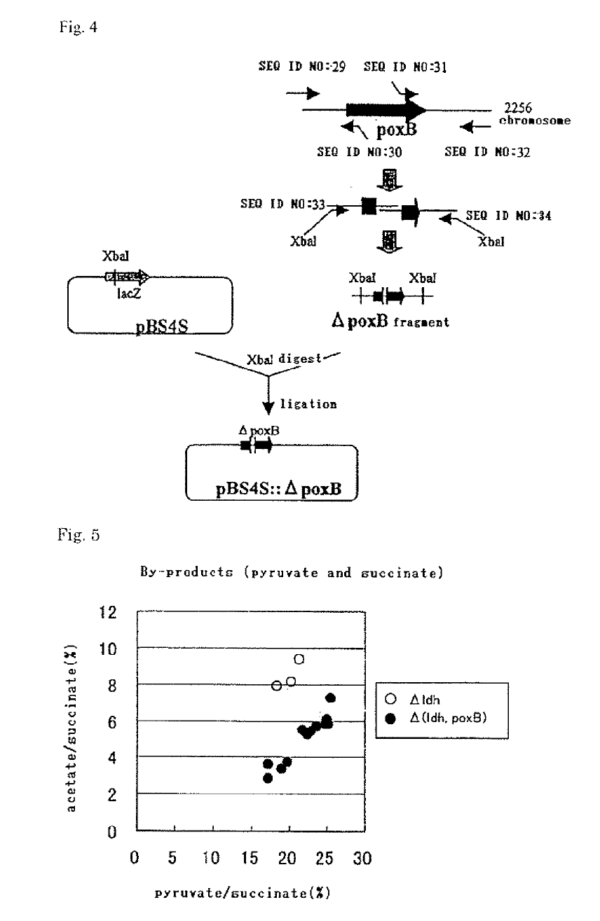

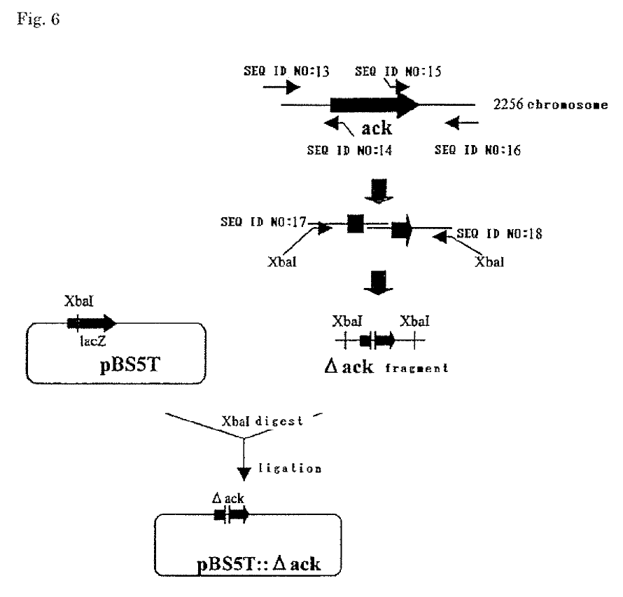

The obtained PCR product was purified by a conventional procedure and then digested with XbaI, followed by insertion into XbaI site of pBS5T constructed in the above-mentioned Example 1(C). Competent cells of Escherichia coli JM109 (Takara Bio Inc.) were used for transformation with this DNA and applied on an LB medium containing 100 μM of IPTG, 40 μg/ml of X-Gal, and 25 μg/ml of kanamycin, followed by overnight culture. Subsequently, appeared white colonies were picked up, and single colonies were isolated, thereby transformants were obtained. Plasmids were extracted from the transformants, and a plasmid into which a PCR product of interest was inserted was named pBS5T::Δack. FIG. 6 shows the construction procedures of pBS5T::Δack.

(B) Preparation of ack-disrupted Strain

The replication origin for coryneform bacteria in pBS5T::Δack obtained in the above-mentioned (A) is temperature-sensitive. That is, the plasmid is autonomously replicable in a cell of a coryneform bacterium at 25° C., but it is not autonomously replicable at 31.5° C. (or 34° C.) Brevibacterium lactofermentum 2256Δ(ldh) strain was transformed using the plasmid by the electric pulse method, and applied on a CM-Dex medium containing 25 μg/ml of kanamycin, followed by culture at 25° C. for 2 nights. Appeared colonies were isolated, to thereby yield transformants. The transformants contain the plasmid. The transformants were cultured at 34° C. overnight in the CM-Dex medium not containing kanamycin and then, after suitable dilution, it was applied on a CM-Dex medium containing 25 μg/ml of kanamycin, followed by culture at 34° C. for about 30 hours. The strain grown on the medium contains a kanamycin resistance gene and a sacB gene which are derived from the plasmid on the genome, as a result of homologous recombination between the ack gene fragment on the plasmid and the ack gene on a genome of Brevibacterium lactofermentum 2256Δ(ldh) strain.

next, the single crossover recombinant was cultured at 31.5° C. overnight in a CM-Dex liquid medium not containing kanamycin and, after suitable dilution, it was applied on 10% sucrose-containing Dex-S10 medium not containing kanamycin, followed by culture at 31.5° C. for about 30 hours. As a result, about 50 strains, which were considered to become sucrose-insensitive due to elimination of the sacB gene by the second homologous recombination, were obtained.

The thus obtained strains include: a strain in which ack gene was replaced by the mutant type derived from pBS5T::Δack; and a strain in which ack gene reverted to the wild type. Whether the ack gene is the mutant type or the wild type can be confirmed easily by directly subjecting a bacterial strain obtained through culturing in a Dex-S10 agar medium to PCR and detecting the ack gene. Analysis of the ack gene by using primers (SEQ ID NOS: 13 and 16) for PCR amplification should result in a DNA fragment of 3.7 kb for the wild type and a DNA fragment of 2.5 kb for the mutant type having the deleted region. As a result of the analysis of the sucrose-insensitive strain by the above-mentioned method, a strain carrying only the mutant type gene was selected and named 2256Δ(ldh, ack).

(5-2) <Construction of Acetate Kinase Gene- and Phosphotransacetylase Gene-disrupted Strain>

(A) Cloning of Fragments for Disrupting Acetate Kinase Gene and Phosphotransacetylase Gene

The ORFs of acetate kinase (ack) gene and phosphotransacetylase gene (hereinafter, referred to as pta) of Brevibacterium lactofermentum 2256 strain have an operon structure, and the both ORFs can be made deficient simultaneously. These gene fragments were obtained by cross-over PCR using as primers synthetic DNAs designed based on the nucleotide sequences of the genes of Corynebacterium glutamicum ATCC13032 (NCgl2656 and 2657 of GenBank Database Accession No. NC_{—}003450; SEQ ID NO: 45), which have already been disclosed. That is, PCR was carried out using a genomic DNA of Brevibacterium lactofermentum 2256 strain as a template and synthetic DNAs of SEQ ID NOS: 19 and 20 as primers, to thereby yield an amplified product of N-terminal region of the pta gene. On the other hand, to yield an amplified product of C-terminal region of the ack gene, PCR was carried out using a genomic DNA of Brevibacterium lactofermentum 2256 strain as a template and synthetic DNAs of SEQ ID NOS: 21 and 16 as primers. SEQ ID NOS: 20 and 21 are partially complementary to each other. The PCR was performed by using KOD-plus-(TOYOBO) in such a way that one cycle of heat-retention at 94° C. for 2 minutes was performed and then, for the N-terminal region, a cycle of denaturation 94° C. for 10 seconds, annealing at 55° C. for 30 seconds and elongation at 68° C. for 30 seconds, and for the C-terminal region, a cycle of denaturation at 94° C. for 10 seconds, annealing at 55° C. for 30 seconds and elongation at 68° C. for 2 minutes were each repeated 30 times, respectively.

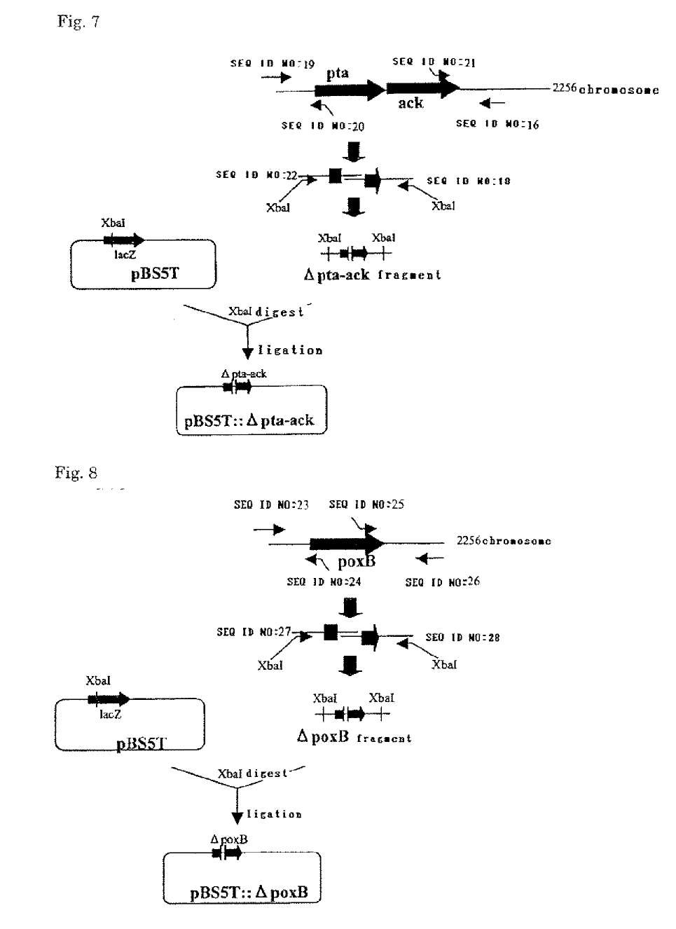

Next, to obtain a fragment of a pta-ack gene in which an internal sequence in pta and ack is deleted, the gene products of the above-mentioned N-terminal region of pta and C-terminal region of ack were mixed at an approximately equimolar concentration, and PCR was carried out using the mixture as templates and synthetic DNAs of SEQ ID NOS: 22 and 18 as primers, to thereby yield amplified product of a mutation-introduced pta-ack gene. The PCR was carried out using KOD-plus-(TOYOBO) in such a way that one cycle of heat-retention at 94° C. for 2 minutes was performed and then a cycle of denaturation at 94° C. for 10 seconds, annealing at 55° C. for 30 seconds and elongation at 68° C. for 2.5 minutes was repeated 30 times, to thereby yield an amplified product of the mutation-introduced pta-ack gene of interest. The PCR product thus obtained was purified by a conventional procedure and digested with XbaI, followed by insertion into XbaI site of pBS5T. Competent cells of Escherichia coli JM109 (Takara Bio Inc.) were used for transformation with this DNA and applied on an LB medium containing 100 μM of IPTG, 40 μg/ml of X-Gal, and 25 μg/ml of kanamycin, followed by overnight culture. Subsequently, appeared white colonies were picked up, and single colonies were isolated, thereby transformants were obtained. Plasmids were extracted from the transformants, and a plasmid into which PCR product of interest was inserted was named pBS5T::Δpta-ack. FIG. 7 shows the construction procedures of pBS5T::Δpta-ack.

(B) Preparation of pta-ack-disrupted Strain

The replication origin for coryneform bacteria in pBS5T::Δpta-ack obtained in the above-mentioned (A) is temperature-sensitive. That is, the plasmid is autonomously replicable in a cell of a coryneform bacterium at 25° C., but it is not autonomously replicable at 31.5° C. (or 34° C.). Brevibacterium lactofermentum 2256Δ(ldh) strain was transformed using the plasmid by the electric pulse method, and applied on a CM-Dex medium containing 25 μg/ml of kanamycin, followed by culture at 25° C. for 2 nights. Appeared colonies were isolated, to thereby yield transformants. The transformants have the plasmid. The transformants were cultured at 34° C. overnight in a CM-Dex liquid medium not containing kanamycin and then, after suitable dilution, it was applied on a CM-Dex liquid medium containing 25 μg/ml of kanamycin, followed by culture at 34° C. for about 30 hours. The strain grown on the medium contains the kanamycin resistance gene and sacB gene which are derived from the plasmid on the genome, as a result of homologous recombination between the pta-ack gene fragment on the plasmid and the pta-ack gene on a genome of Brevibacterium lactofermentum 2256Δ(ldh) strain.

Next, the single crossover recombinant was cultured at 31.5° C. overnight in CM-Dex liquid medium not containing kanamycin and, after suitably dilution, it was applied on 10% sucrose-containing Dex-S10 medium not containing kanamycin, followed by culture at 31.5° C. for about 30 hours. As a result, about 50 strains, which were considered to become sucrose-insensitive due to elimination of the sacB gene by the second homologous recombination, were obtained.

The thus obtained strains include: a strain in which pta and ack genes were replaced by the mutant type derived from pBS5T::Δpta-ack; and a strain in which pta and ack genes reverted to the wild type. Whether the pta and ack genes is the mutant type or the wild type can be confirmed easily by directly subjecting a bacterial strain obtained through culture in a Dex-S10 agar medium to PCR and detecting the pta and ack genes. Analysis of the pta-ack gene by using primers (SEQ ID NOS: 19 and 16) for PCR amplification should result in a DNA fragment of 5.0 kb for the wild type and a DNA fragment of 2.7 kb for the mutant type having a deleted region.

As a result of the analysis of the sucrose-insensitive strains by the above-mentioned method, a strain carrying only the mutant type gene was selected and named 2256Δ(ldh, pta, ack).

(5-3) <Construction of Acetate Kinase Gene-, Phosphotransacetylase Gene-, Pyruvate Oxidase Gene-disrupted Strain>

(A) Cloning of a Fragment for Disrupting Pyruvate Oxidase Gene

A fragment of a pyruvate oxidase gene (hereinafter, abbreviated as poxB) of Brevibacterium lactofermentum 2256 strain in which the ORF thereof was deleted was obtained by crossover PCR using as primers synthetic DNAs designed based on the nucleotide sequence of the gene of Corynebacterium glutamicum ATCC13032 (NCgl2521 of GeneBank Database Accession No. NC_{—}003450; SEQ ID NO: 48), which has already been disclosed. That is, PCR was carried out using a genomic DNA of Brevibacterium lactofermentum 2256 strain as a template and synthetic DNAs of SEQ ID NOS: 23 and 24 as primers, thereby amplified product of N-terminal region of the poxB gene was obtained.

On the other hand, to obtain amplified product of C-terminal region of the poxB gene, PCR was carried out using a genomic DNA of Brevibacterium lactofermentum 2256 strain as a template and synthetic DNAs of SEQ ID NOS: 25 and 26 as primers. SEQ ID NOS: 24 and 25 are complementary to each other. The PCR was carried out using KOD-plus-(TOYOBO) in such a way that one cycle of heat-retention at 94° C. for 2 minutes was performed and then a cycle of denaturation at 94° C. for 10 seconds, annealing at 55° C. for 30 seconds and elongation at 68° C. for 40 seconds was repeated 30 times, for both of the N-terminal region and the C-terminal region. Next, to obtain a fragment of poxB gene in which its internal sequence is deleted, the above-mentioned gene products of the N-terminal and C-terminal regions of poxB were mixed at an approximate equimolar concentration, and PCR was carried out using the mixture as templates and synthetic DNAs of SEQ ID NOS: 27 and 28 as primers, to thereby yield amplified product of a mutation-introduced poxB gene. The PCR was carried out using KOD-plus-(TOYOBO) in such a way that one cycle of heat-retention at 94° C. for 2 minutes was performed, and then a cycle of denaturation at 94° C. for 10 seconds, annealing at 55° C. for 30 seconds and elongation at 68° C. for 70 seconds was repeated 30 times, to thereby yield an amplified product of the mutation-introduced poxB gene of interest.

The PCR product thus obtained was purified by a conventional procedure and then digested with XbaI, followed by insertion into XbaI site of pBS5T constructed in the above-mentioned Example 1 (C). Competent cells of Escherichia coli JM109 (Takara Bio Inc.) were used for transformation with this DNA and applied on an LB medium containing 100 μM of IPTG, 40 μg/ml of X-Gal, and 25 μg/ml of kanamycin, followed by overnight culture. Subsequently, appeared white colonies were picked up, and single colonies were isolated, thereby transformants were obtained. Plasmids were extracted from the transformants, and a plasmid into which a PCR product of interest was inserted was named pBS5T::ΔpoxB. FIG. 8 shows the construction procedures of pBS5T::ΔpoxB.

(B) Preparation of poxB-disrupted Strain

The replication origin for coryneform bacteria in pBS5T::ΔpoxB obtained in the above-mentioned Example 5 (A) is temperature-sensitive. That is, the plasmid is autonomously replicable in a cell of a coryneform bacterium at 25° C., but it is not autonomously replicable at 31.5° C. (or 34° C.). Brevibacterium lactofermentum 2256Δ(ldh, pta, ack) strain was transformed using the plasmid by the electric pulse method, and then applied on a CM-Dex medium containing 25 μg/ml of kanamycin, followed by culture at 25° C. for 2 nights. Appeared colonies were isolated, to thereby yield transformants. The transformants should have the plasmid.

The transformants were cultured at 34° C. overnight in a CM-Dex liquid medium not containing kanamycin, and after suitable dilution, it was applied on a CM-Dex medium containing 25 μg/ml of kanamycin, followed by culture at 34° C. for about 30 hours. In the strain grown on the medium, the kanamycin resistance gene and sacB gene which are derived from the plasmid are inserted into the genome, as a result of homologous recombination between the poxB gene fragment on the plasmid and the poxB gene on a genome of Brevibacterium lactofermentum 2256Δ(ldh, pta, ack) strain.

Next, the single crossover recombinant was cultured at 31.5° C. overnight in CM-Dex liquid medium not containing kanamycin, and after suitable dilution, it was applied on 10% sucrose-containing Dex-S10 medium not containing kanamycin, followed by culture at 31.5° C. for about 30 hours. As a result, about 50 strains, which were considered to become sucrose-insensitive due to elimination of the sacB gene by the second homologous recombination, were obtained.

The thus obtained strains include: a strain in which poxB gene was replaced by the mutant type derived from pBS5T::ΔpoxB; and a strain in which the poxB gene reverted to the wild type. Whether the poxB gene is the mutant type or the wild type can be confirmed easily by directly subjecting a bacterial strain obtained through culture in a Dex-S10 agar medium to PCR and detecting the poxB gene. By analyzing the poxB gene by using primers (SEQ ID NOS: 23 and 26) for PCR amplification, A DNA fragment of 2.4 kb for the wild type and a DNA fragment of 1.2 kb for the mutant type having the deleted region can be detected. As a result of the analysis of the sucrose-insensitive strain by the above-mentioned method, a strain carrying only the mutant type gene was selected and named 2256Δ(ldh, pta, ack, poxB). The strain is also called a pta, ack, poxB-disrupted strain, herein.

(5-4) <Construction of poxB, pta, ack ach Gene-disrupted Strain>

(A) Cloning of a Fragment for Disrupting Acetyl-CoA Hydrolase Gene

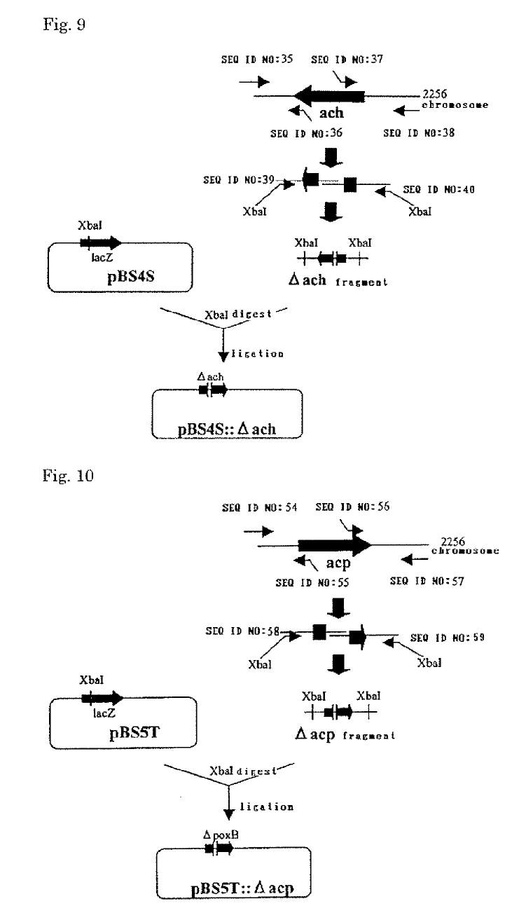

A fragment of an acetyl-CoA hydrolase gene (hereinafter, abbreviated as ach) of Brevibacterium lactofermentum 2256 strain in which ORF thereof was deleted was obtained by crossover PCR using as primers synthetic DNAs designed based on the nucleotide sequence of the gene of Corynebacterium glutamicum ATCC13032 (NCgl2480 of GenBank Database Accession No. NC_{—}003450; SEQ ID NO: 50), which has already been disclosed. That is, PCR was carried out using a genomic DNA of Brevibacterium lactofermentum 2256 strain as a template and synthetic DNAs of SEQ ID NOS: 35 and 36 as primers, thereby amplified product of C-terminal region of the ach gene was obtained. On the other hand, to obtain amplified product of N-terminal region of the ach gene, PCR was carried out using a genomic DNA of Brevibacterium lactofermentum 2256 strain as a template and synthetic DNAs of SEQ ID NOS: 37 and 38 as primers. SEQ ID NOS: 37 and 38 are complementary to each other. The PCR was carried out using KOD-plus-(TOYOBO) in such a way that one cycle of heat-retention at 94° C. for 2 minutes was performed and then a cycle of denaturation at 94° C. for 10 seconds, annealing at 55° C. for 30 seconds and elongation at 68° C. for 50 seconds was repeated 30 times, for the N-terminal region and the C-terminal region. Next, to obtain a fragment of the ach gene in which its internal sequence is deleted, the above-mentioned gene products of the N-terminal and C-terminal regions of ach were mixed at an approximately equimolar concentration, and PCR was carried out using the mixture as templates and synthetic DNAs of SEQ ID NOS: 39 and 40 as primers, to thereby yield amplified products of a mutation-introduced ach gene. The PCR was carried out using KOD-plus-(TOYOBO) in such a way that one cycle of heat-retention at 94° C. for 2 minutes was performed and then a cycle of denaturation at 94° C. for 10 seconds, annealing at 55° C. for 30 seconds and elongation at 68° C. for 90 seconds was repeated 30 times, to thereby yield an amplified product of the mutation-introduced ach gene of interest. The PCR product thus obtained was purified by a conventional procedure and digested with XbaI, followed by insertion into XbaI site of pBS4S constructed in the above-mentioned Example 1 (B). Competent cells of Escherichia coli JM109 (Takara Bio Inc.) were used for transformation with this DNA and applied on an LB medium containing 100 μM of IPTG, 40 μg/ml of X-Gal, and 25 μg/ml of kanamycin, followed by overnight culture. Subsequently, appeared white colonies were picked up, and single colonies were isolated, thereby transformants were obtained. Plasmids were extracted from the transformants, and a plasmid into which a PCR product of interest was inserted was named pBS4S::Δach. FIG. 9 shows the construction procedures of pBS4S::Δach.

(B) Preparation of ach-disrupted Strain

The pBS4S::Δach obtained in the above-mentioned (A) does not include a region which enables autonomous replication in a cell of coryneform bacterium, so when a coryneform bacterium is transformed with the plasmid, a strain in which the plasmid is integrated into a chromosome by homologous recombination appears at a very low frequency as a transformant. Brevibacterium lactofermentum 2256Δ(ldh, pta, ack, poxB) strain and 2256 strain were transformed by using a high concentration of the plasmid pBS4S::Δach by the electric pulse method, and applied on a CM-Dex medium containing 25 μg/ml kanamycin, followed by culture at 31.5° C. for about 30 hours. In the strains grown on the medium, a kanamycin resistance gene and a sacB gene derived from the plasmid are inserted on the genome, as a result of homologous recombination between the ach gene fragment on the plasmid and the ach gene on a genome of each of Brevibacterium lactofermentum 2256Δ(ldh) strain, 2256Δ(ldh, pta, ack) strain, 2256Δ(ldh, pta, ack, poxB) strain, and 2256Δ(ldh, pta, ack, poxB, acp) strain.

Next, the single crossover recombinant was cultured at 31.5° C. overnight in CM-Dex liquid medium not containing kanamycin and then, after suitable dilution, it was applied on 10% sucrose-containing Dex-S10 medium not containing kanamycin, followed by culture at 31.5° C. for about 30 hours. As a result, about 50 strains, which were considered to become sucrose-insensitive due to elimination of the sacB gene by the second homologous recombination, were obtained.

The thus obtained strains include: a strain in which ach gene was replaced by the mutant type derived from pBS4S::Δach; and a strain in which ach gene reverted to the wild type. Whether the ach gene is the mutant type or the wild type can be confirmed easily by directly subjecting a bacterial strain obtained through culture in a Dex-S10 agar medium to PCR and detecting the ach gene. Analysis of the ach gene by using primers (SEQ ID NOS: 35 and 38) for PCR amplification should result in a DNA fragment of 2.9 kb for the wild type and a DNA fragment of 1.4 kb for the mutant type having the deleted region. As a result of the analysis of the sucrose-insensitive strain by the above-mentioned method, a strain-carrying only the mutant type gene was selected and the strain obtained from 2256Δ(ldh, pta, ack, poxB) was named 2256Δ(ldh, pta, ack, poxB, ach) strain. The strain is also called an ach, pta, ack, poxB-disrupted strain, herein.

### Example 6

### Construction of Acylphosphatase Gene-disrupted Strain

(A) Cloning of a Fragment for Disrupting Acylphosphatase Gene

A gene fragment of an acylphosphatase gene (hereinafter, referred to as acp) of Brevibacterium lactofermentum 2256 strain in which ORF thereof was deleted was obtained by crossover PCR using as primers synthetic DNAs designed based on a nucleotide sequence (SEQ ID NO. 52), which is obtained as a sequence having high homology to acp gene of Mycobacterium tuberculos from a search in a sequence of Brevibacterium lactofermentum ATCC13869 strain as identified by genomic analysis. That is, PCR was carried out using a genomic DNA of Brevibacterium lactofermentum 2256 strain as a template and synthetic DNAs of SEQ ID NOS: 54 and 55 as primers, thereby an amplified product of C-terminal region of the acp gene was obtained. On the other hand, to obtain an amplified product of N-terminal region of the acp gene, PCR was carried out using a genomic DNA of Brevibacterium lactofermentum 2256 strain as a template and synthetic DNAs of SEQ ID NOS. 56 and 57 as primers. SEQ ID NOS: 55 and 56 are complementary to each other. The PCR was carried out using KOD-plus-(TOYOBO) in such a way that one cycle of heat-retention at 94° C. for 2 minutes was performed and then a cycle of denaturation at 94° C. for 10 seconds, annealing at 55° C. for 30 seconds and elongation at 68° C. for 35 seconds was repeated 30 times for the N-terminal and C-terminal regions. Next, to obtain a fragment of an acp gene with a deletion of an internal sequence, the above-mentioned amplified products of the N-terminal and C-terminal regions of acp were mixed at an approximately equimolar concentration, and PCR was carried out using the mixture as templates and synthetic DNAs of SEQ ID NOS. 58 and 59 as primers, to thereby yield an amplified product of a mutation-introduced acp gene. The PCR was carried out using KOD-plus-(TOYOBO) in such a way that one cycle of heat-retention at 94° C. for 2 minutes was performed and then a cycle of denaturation at 94° C. for 10 seconds, annealing at 55° C. for 30 seconds and elongation at 68° C. for 60 seconds was repeated 30 times, to thereby obtain an amplified product of the mutation-introduced acp gene of interest.

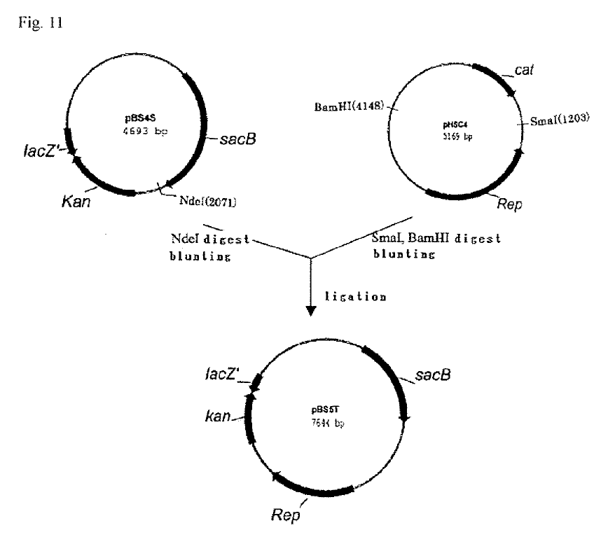

The PCR product thus obtained was purified by a conventional procedure and then digested with XbaI, followed by insertion into XbaI site of pBS5T constructed in the above-mentioned Example 1 (C). Competent cells of Escherichia coli JM109 (Takara Bio Inc.) were used for transformation with this DNA and applied on an LB medium containing 100 μM IPTG, 40 μg/ml X-Gal, and 25 μg/ml kanamycin, followed by overnight culture. Subsequently, appeared white colonies were picked up, and single colonies were isolated, thereby transformants were obtained. Plasmids were extracted from the transformants, and a plasmid into which a PCR product of interest was inserted was named pBS5T::Δacp. FIG. 10 shows the construction procedures of pBS5T::Δacp.

(B) Preparation of acp-disrupted Strain

The pBS5T::Δacp obtained in the above-mentioned (A) does not include a region which enables autonomous replication in a cell of a coryneform bacterium, so when a coryneform bacterium is transformed with the plasmid, a strain in which the plasmid is integrated into a chromosome by homologous recombination appears at a very low frequency as a transformant. Brevibacterium lactofermentum 2256Δ(ldh, pta, ack, poxB) strain was transformed by using a high concentration of the plasmid pBS5T::Δacp by the electric pulse method, and applied on a CM-Dex medium containing 25 μg/ml kanamycin, followed by culture at 31.5° C. for about 30 hours. In the strain grown on the medium, the kanamycin resistance gene and sacB gene derived from the plasmid are inserted on the genome, as a result of homologous recombination between the acp gene fragment on the plasmid and the acp gene on a genome of Brevibacterium lactofermentum 2256Δ(ldh, pta, ack, poxB) strain.

Next, the single crossover recombinant was cultured at 31.5° C. overnight in CM-Dex liquid medium not containing kanamycin and, after suitable dilution, it was applied on 10% sucrose-containing Dex-S10 medium not containing kanamycin, followed by culture at 31.5° C. for about 30 hours. As a result, about 50 strains, which were considered to become sucrose-insensitive due to elimination of the sacB gene by the second homologous recombination, were obtained.

The thus obtained strains include: a strain in which acp gene was replaced by the mutant type derived from pBS5T::Δacp; and a strain in which acp gene reverted to the wild type. Whether the acp gene is the mutant type or the wild type can be confirmed easily by directly subjecting a bacterial strain obtained through culture in a Dex-S10 agar medium to PCR and detecting the acp gene. Analysis of the acp gene by using primers (SEQ ID NOS: 54 and 57) for PCR amplification should result in a DNA fragment of 1.3 kb for the wild type and a DNA fragment of 1.0 kb for the mutant type having the deleted region. As a result of the analysis of the sucrose-insensitive strain by the above-mentioned method, a strain carrying only the mutant type gene was selected and named 2256Δ(ldh, pta, ack, poxB, acp) strain.

### Example 7

### <7-1> Evaluation of Culture of ach, pta, ack, poxB-disputed Strain and pta, ack, poxB-disputed Strain

Brevibacterium lactofermentum 2256Δ(ldh) strain, 2256Δ(ldh, pta-ack, poxB) strain, and 2256Δ(ldh, pta-ack, poxB, ach) strain were used for culture for producing succinic acid as described above. The bacterial cells of the 2256Δ(ldh) strain, 2256Δ(ldh, pta-ack, poxB) strain, and 2256Δ (ldh, pta-ack, poxB, ach) strain obtained by culturing them on a CM-Dex plate medium were inoculated into 3 ml of a seed medium B (10 g/L of glucose, 2.5 g/L of (NH₄)₂SO₄, 0.5 g/L of KH₂PO₄, 0.25 g/L of MgSO₄.7H₂O, 2 g/L of urea, 0.01 g/L of FeSO₄.7H₂O, 0.01 g/L of MnSO₄.7H₂O, 50 μg/L of biotin, 100 μg/L of VB1.HCl, 15 mg/L of protocatechuic acid, 0.02 mg/L of CuSO₄, and 10 mg/l of CaCl₂, with pH 7.0 (KOH)). Shaking culture was performed in a test tube at 31.5° C. for about 15 hours under an aerobic condition.

After that, 3 ml of a main medium B (70 g/L of glucose, 5 g/L of (NH₄)₂SO₄, 2 g/L of KH₂PO₄, 3 g/L of urea, 0.01 g/L of FeSO₄.7H₂O, 0.01 g/l of MnSO₄.7H₂O, 200 μg/L of biotin, 200 μg/L of VB1.HCl, 40 g/L of MOPS, and 50 g/L of MgCO₃, with pH 6.8 NaOH)) was added into the tube. For preventing aeration, the succinic acid production culture was carried out while the tube was sealed hermetically with a silicon cap. The culture was performed by shaking at 31.5° C. for about 24 hours and terminated before sugar in the medium was exhausted. After completion of the culture, the accumulation amounts of succinic acid and by-product acetic acid in the medium were analyzed by liquid chromatography after the medium had been suitably diluted. A column obtained by connecting two pieces of Shim-pack SCR-102H (Shimadzu) in series was used, and the sample was eluted at 40° C. by using 5 mM p-toluene sulfonic acid. The eluent was neutralized by using 20 mM Bis-Tris aqueous solution containing 5 mM p-toluene sulfonic acid and 100 μM of EDTA. The succinic acid and acetic acid were each measured by determining the electric conductivity by means of CDD-10AD (Shimadzu). The obtained results are shown in Table 2.

The acetic acid in the 2256Δ(ldh, pta-ack, poxB) strain was drastically decreased as compared to the control 2256Δldh strain, and the acetic acid in the 2256Δ(ldh, pta, ack, ach, poxB) strain was further reduced, that is, reduced by about 40% in comparison with the 2256Δ(ldh, pta, ack, poxB) strain. These results revealed that eliminating or decreasing all or any one of the activities of poxB, pta-ack and ach simultaneously is effective for reducing acetic acid.

TABLE 2
Production of succinic acid and acetic acid in the strains in which
ACH, PTA, ACK and POXB are disrupted in combination

| | | | Yield of | Acetic acid |
|---|---|---|---|---|
| | | Consumed | succinic acid | (/succinic |
| Strains | OD620(x51) | sugar (g/L) | (%) | acid %) |
| 2256Δldh | 0.342 | 31.8 | 57.3 | 11.9 |
| 2256Δ(ldh, pta, | 0.382 | 36.6 | 56.2 | 8.4 |
| ack, poxB) | | | | |
| 2256Δ(ldh, pta, | 0.372 | 39.8 | 56.1 | 3.6 |
| ack, poxB, ach) | | | | |

<7-2> Evaluation of Culture of the pta, ack, poxB, acp-disrupted Strain

Brevibacterium lactofermentum 2256Δ(ldh) strain, 2256Δ(ldh, pta, ack, poxB) strain, and 2256Δ(ldh, pta-ack, poxB, acp) strain were used for culture for producing succinic acid as follows. The bacterial cells of the 2256Δ(ldh) strain, 2256Δ(ldh, pta, ack poxB) strain and 2256Δ(ldh, pta-ack, poxB, acp) strain obtained by culturing them on a CM-Dex plate medium were inoculated into 3 ml of the above-mentioned seed medium B. Shaking culture was performed in a test tube at 31.5° C. for about 15 hours under an aerobic condition.

After that, 3 ml of the above-mentioned main medium B was added into the tube. For preventing aeration, the succinic acid production culture was carried out while the tube was sealed hermetically with a silicon cap. The culture was performed by shaking at 31.5° C. for about 24 hours and terminated before sugar in the medium had been exhausted.

After completion of the culture, the accumulation amounts of succinic acid and by-product acetic acid in the culture medium were analyzed by liquid chromatography after the culture medium had been suitably diluted. A column obtained by connecting two pieces of Shim-pack SCR-102Hs (Shimadzu) in series was used, and the sample was eluted at 40° C. by using 5 mM of p-toluene sulfonic acid. The eluent was neutralized by using 20 mM of Bis-Tris aqueous solution containing 5 mM of p-toluene sulfonic acid and 100 μM of EDTA. The succinic acid and by-product acetic acid were each measured by determining the electric conductivity by means of CDD-10AD (Shimadzu). The obtained results are shown in Table 3.

TABLE 3
Production of succinic acid and acetic acid in the strains in which
pta, ack, poxB and acp are disrupted in combination

| | | | Yield of | Acetic acid |
|---|---|---|---|---|
| | OD620 | Consumed | succinic acid | (/succinic |
| Strains | nm(x51) | sugar (g/L) | (%) | acid %) |
| 2256Δldh | 0.333 | 45.9 | 49.2 | 10.4 |
| 2256Δ(ldh, pta, | 0.307 | 40.7 | 45.2 | 8.9 |
| ack, poxB) | | | | |
| 2256Δ(ldh, pta, | 0.341 | 44.3 | 46.0 | 8.8 |
| ack, poxB, acp) | | | | |

As a result, ratio of acetic acid with respect to succinic acid was slightly reduced by decreasing or eliminating all of the activities of PTA, ACK and POXB, even when ACH activity was not decreased or eliminated as in the above-mentioned (C). On the other hand, decreasing or eliminating ACP activity had little influence on production of acetic acid and succinic acid.

### INDUSTRIAL APPLICABILITY

The present invention is useful for fermentative production of succinic acid. Succinic acid is useful as a raw material for biodegradable polymers, food products, drugs, cosmetics, and the like.

While the invention has been described in detail with reference to preferred embodiments thereof, it will be apparent to one skilled in the art that various changes can be made, and equivalents employed, without departing from the scope of the invention. Each of the aforementioned documents, including the foreign priority document, JP 2004-150672, is incorporated by reference herein in its entirety.

1. A coryneform bacterium having a succinic acid-producing ability, wherein said bacterium has been modified so that an activity of pyruvate oxidase is decreased.

2. The coryneform bacterium according to claim 1, wherein the pyruvate oxidase is a protein as described in the following (A) or (B):
(A) a protein having an amino acid sequence of SEQ ID NO: 49; or
(B) a protein having an amino acid sequence of SEQ ID NO: 49 including substitution, deletion, insertion, or addition of one or several amino acids, and having a pyruvate oxidase activity.

3. The coryneform bacteria according to claim 1, wherein the pyruvate oxidase activity is decreased by disruption of a pyruvate oxidase gene on a chromosome.

4. The coryneform bacterium according to claim 3, wherein the pyruvate oxidase gene is a DNA as described in the following (a) or (b);
(a) a DNA comprising the nucleotide sequence of nucleotide numbers 996-2732 in SEQ ID NO: 48; or
(b) a DNA that hybridizes with the nucleotide sequence of nucleotide numbers 996-2732 in SEQ ID NO: 48 or a probe that can be prepared from the nucleotide sequence under stringent conditions, and encodes a protein having a pyruvate oxidase activity.

5. The coryneform bacterium according to claim 1, wherein said bacterium has been further modified so that activities of one or more of phosphotransacetylase, acetate kinase and acetyl-CoA Hydrolase is decreased.

6. The coryneform bacterium according to claim 1, wherein said bacterium has been further modified so that an activity of lactate dehydrogenase is decreased.

7. The coryneform bacterium according to claim 1, wherein said bacterium has been further modified so that an activity of pyruvate carboxylase is increased.

8. A method for producing succinic acid, comprising:
allowing the coryneform bacterium according to claim 1 or a treated product thereof to act on an organic raw material in a reaction liquid containing carbonate ion, bicarbonate ion or carbon dioxide to produce and accumulate succinic acid in the reaction liquid; and
collecting succinic acid from the reaction liquid.

9. The production method according to claim 8, wherein the bacterium or treated product thereof is allowed to act on the organic raw material under anaerobic conditions.

10. A method for producing a succinic acid-containing polymer, comprising the steps of producing succinic acid by the method according to claim 8 and polymerizing the obtained succinic acid.


SEQUENCE LISTING (61 sequences)

SEQ ID NO: 1 - DNA, 24 bp, Artificial Sequence
  Description of Artificial Sequence Synthetic primer
cgggatcctttttaacccatcaca

SEQ ID NO: 2 - DNA, 29 bp, Artificial Sequence
  Description of Artificial Sequence Synthetic primer
gaagatcttcaaaaggttaggaatacggt

SEQ ID NO: 3 - DNA, 23 bp, Artificial Sequence
  Description of Artificial Sequence Synthetic primer
ccttttgaagatcgaccagttgg

SEQ ID NO: 4 - DNA, 44 bp, Artificial Sequence
  Description of Artificial Sequence Synthetic primer
tacctggaatgctgttttcccagggatcgcagtggtgagtaacc

SEQ ID NO: 5 - DNA, 28 bp, Artificial Sequence
  Description of Artificial Sequence Synthetic primer
cctgggaaaacagcattccaggtattag

SEQ ID NO: 6 - DNA, 23 bp, Artificial Sequence
  Description of Artificial Sequence Synthetic primer
tgcaggtcgactctagaggatcc

SEQ ID NO: 7 - DNA, 20 bp, Artificial Sequence
  Description of Artificial Sequence Synthetic primer
cactgcacggccctgcgaac

SEQ ID NO: 8 - DNA, 42 bp, Artificial Sequence
  Description of Artificial Sequence Synthetic primer
cgccaactaggcgccaaaaattcctgatttccctaaccggac

SEQ ID NO: 9 - DNA, 42 bp, Artificial Sequence
  Description of Artificial Sequence Synthetic primer
gtccggttagggaaatcaggaatttttggcgcctagttggcg

SEQ ID NO: 10 - DNA, 20 bp, Artificial Sequence
  Description of Artificial Sequence Synthetic primer
tgtgggccttcggcgaggac

SEQ ID NO: 11 - DNA, 26 bp, Artificial Sequence
  Description of Artificial Sequence Synthetic primer
gagtcgaccgcaccccatttttcata

SEQ ID NO: 12 - DNA, 28 bp, Artificial Sequence
  Description of Artificial Sequence Synthetic primer
tggtcgacgtgaatgctcggcgggatcc

SEQ ID NO: 13 - DNA, 24 bp, Artificial Sequence
  Description of Artificial Sequence Synthetic primer
cttccatcttcctcatggtgctgc

SEQ ID NO: 14 - DNA, 44 bp, Artificial Sequence
  Description of Artificial Sequence Synthetic primer
ccaggagagctaagcgaactccattagctgcgtcctcctgcctg

SEQ ID NO: 15 - DNA, 44 bp, Artificial Sequence
  Description of Artificial Sequence Synthetic primer
caggcaggaggacgcagctaatggagttcgcttagctctcctgg

SEQ ID NO: 16 - DNA, 31 bp, Artificial Sequence
  Description of Artificial Sequence Synthetic primer
gcgtctagacctttaggagtgcgatgtcccc

SEQ ID NO: 17 - DNA, 33 bp, Artificial Sequence
  Description of Artificial Sequence Synthetic primer
gcgtctagacgactgtgctgttaacccgaaccc

SEQ ID NO: 18 - DNA, 33 bp, Artificial Sequence
  Description of Artificial Sequence Synthetic primer
gcgtctagagagttaggcccttagaagcgattc

SEQ ID NO: 19 - DNA, 24 bp, Artificial Sequence
  Description of Artificial Sequence Synthetic primer
gctcaaagcgtggaattgagatcg

SEQ ID NO: 20 - DNA, 42 bp, Artificial Sequence
  Description of Artificial Sequence Synthetic primer
ccaggagagctaagcgaactttcggcgctcatgactggttcg

SEQ ID NO: 21 - DNA, 42 bp, Artificial Sequence
  Description of Artificial Sequence Synthetic primer
cgaaccagtcatgagcgccgaaagttcgcttagctctcctgg

SEQ ID NO: 22 - DNA, 30 bp, Artificial Sequence
  Description of Artificial Sequence Synthetic primer
gcgtctagagtacgcaaggcggacgaacgc

SEQ ID NO: 23 - DNA, 23 bp, Artificial Sequence
  Description of Artificial Sequence Synthetic primer
gccttgatatcttcccgcaaacc

SEQ ID NO: 24 - DNA, 47 bp, Artificial Sequence
  Description of Artificial Sequence Synthetic primer
cttgtggtcctggaaacacacaccgaagtgaatttcgcagagattgc

SEQ ID NO: 25 - DNA, 48 bp, Artificial Sequence
  Description of Artificial Sequence Synthetic primer
cgcaatctctgcgaaattcacttcggtgtgtgtttccaggaccacaag

SEQ ID NO: 26 - DNA, 22 bp, Artificial Sequence
  Description of Artificial Sequence Synthetic primer
ggtttctcggggtctaaaccgg

SEQ ID NO: 27 - DNA, 33 bp, Artificial Sequence
  Description of Artificial Sequence Synthetic primer
gggaatctagaccacgccaatggaaatttctcc

SEQ ID NO: 28 - DNA, 35 bp, Artificial Sequence
  Description of Artificial Sequence Synthetic primer
gggaatctagacgtgacaagatctggcgaaatcgc

SEQ ID NO: 29 - DNA, 23 bp, Artificial Sequence
  Description of Artificial Sequence Synthetic primer
gccttgatatcttcccgcaaacc

SEQ ID NO: 30 - DNA, 47 bp, Artificial Sequence; identical to SEQ ID NO: 24
  Description of Artificial Sequence Synthetic primer

SEQ ID NO: 31 - DNA, 48 bp, Artificial Sequence; identical to SEQ ID NO: 25
  Description of Artificial Sequence Synthetic primer

SEQ ID NO: 32 - DNA, 22 bp, Artificial Sequence
  Description of Artificial Sequence Synthetic primer
ggtttctcggggtctaaaccgg

SEQ ID NO: 33 - DNA, 33 bp, Artificial Sequence; identical to SEQ ID NO: 27
  Description of Artificial Sequence Synthetic primer

SEQ ID NO: 34 - DNA, 35 bp, Artificial Sequence; identical to SEQ ID NO: 28
  Description of Artificial Sequence Synthetic primer

SEQ ID NO: 35 - DNA, 24 bp, Artificial Sequence
  Description of Artificial Sequence Synthetic primer
gcttctgcgcaaagcaagcctccg

SEQ ID NO: 36 - DNA, 50 bp, Artificial Sequence
  Description of Artificial Sequence Synthetic primer
gtccgattacctgaggaggtattcccatgaaggcataagttttttcttgg

SEQ ID NO: 37 - DNA, 50 bp, Artificial Sequence
  Description of Artificial Sequence Synthetic primer
ccaagaaaaaacttatgccttcatgggaatacctcctcaggtaatcggac

SEQ ID NO: 38 - DNA, 26 bp, Artificial Sequence
  Description of Artificial Sequence Synthetic primer
ggtcatgtgcatggttttctcattgc

SEQ ID NO: 39 - DNA, 33 bp, Artificial Sequence
  Description of Artificial Sequence Synthetic primer
ggcctctagacctgcaccgatcaggatgagtgg

SEQ ID NO: 40 - DNA, 35 bp, Artificial Sequence
  Description of Artificial Sequence Synthetic primer
gcgctctagactcaacaagagcacgcgcagtcacc

SEQ ID NO: 41 - DNA, 2014 bp, Bacillus subtilis
  CDS (464)..(1882)
gatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctattggcattttcttttatttcttatcaacataaaggtgaatcccatatgaactatataaaagcaggcaaatggctaaccgtattcctaaccttttgaagatc

SEQ ID NO: 42 - protein, 473 aa, Bacillus subtilis
MNIKKFAKQATVLTFTTALLAGGATQAFAKETNQKPYKETYGISHITRHDMLQIPEQQKNEKYQVPEFDSSTIKNISSAKGLDVWDSWPLQNADGTVANYHGYHIVFALAGDPKNADDTSIYMFYQKVGETSIDSWKNAGRVFKDSDKFDANDSILKDQTQEWSGSATFTSDGKIRLFYTDFSGKHYGKQTLTTAQVNVSASDSSLNINGVEDYKSIFDGDGKTYQNVQQFIDEGNYSSGDNHTLRDPHYVEDKGHKYLVFEANTGTEDGYQGEESLFNKAYYGKSTSFFRQESQKLLQSDKKRTAELANGALGMIELNDDYTLKKVMKPLIASNTVTDEIERANVFKMNGKWYLFTDSRGSKMTIDGITSNDIYMLGYVSNSLTGPYKPLNKTGLVLKMDLDPNDVTFTYSHFAVPQAKGNNVVITSYMTNRGFYADKQSTFAPSFLLNIKGKKTSVVKDSILEQGQLTVNK

SEQ ID NO: 43 - DNA, 2820 bp, Corynebacterium glutamicum
  CDS (898)..(1851)
tgcagaattatgcaagatgcgccgcaacaaaacgcgatcggccaaggtcaaagtggtcaatgtaatgaccgaaaccgctgcgatgaaacttatccacggcggtaaaaacctctcaattaggagcttgacctcattaatactgtgctgggttaattcgccggtgatcagcagcgcgccgtaccccaaggtgccgacactaatgcccgcgatcgtctccttcggtccaaaattcttctgcccaatcagccggatttgggtgcgatgcctgatcaatcccacaaccgtggtggtcaacgtgatggcaccagttgcgatgtgggtggcgttgtaaattttcctggatacccgccggttggttctggggaggatcgagtggattcccgtcgctgccgcatgccccaccgcttgtaaaacagccaggttagcagccgtaacccaccacggtttcggcaacaatgacggcgagagagcccaccacattgcgatttccgctccgataaagccagcgcccatatttgcagggaggattcgcctgcggtttggcgacattcggatccccggaactagctctgcaatgacctgcgcgccgagggaggcgaggtgggtggcaggttttagtgcgggtttaagcgttgccaggcgagtggtgagcagagacgctagtctggggagcgaaaccatattgagtcatcttggcagagcatgcacaattctgcagggcataggttggttttgctcgatttacaatgtgattttttcaacaaaaataacacttggtctgaccacattttcggacataatcgggcataattaaaggtgtaacaaaggaatccgggcacaagctcttgctgattttctgagctgctttgtgggttgtccggttagggaaatcaggaagtgggatcgaaaatgaaagaaaccgtcggtaacaagattgtcctcattggcgcaggagatgttggagttgcatacgcatacgcactgatcaaccagggcatggcagatcaccttgcgatcatcgacatcgatgaaaagaaactcgaaggcaacgtcatggacttaaaccatggtgttgtgtgggccgattcccgcacccgcgtcaccaagggcacctacgctgactgcgaagacgcagccatggttgtcatttgtgccggcgcagcccaaaagccaggcgagacccgcctccagctggtggacaaaaacgtcaagattatgaaatccatcgtcggcgatgtcatggacagcggattcgacggcatcttcctcgtggcgtccaacccagtggatatcctgacctacgcagtgtggaaattctccggcttggaatggaaccgcgtgatcggctccggaactgtcctggactccgctcgattccgctacatgctgggcgaactctacgaagtggcaccaagctccgtccacgcctacatcatcggcgaacacggcgacactgaacttccagtcctgtcctccgcgaccatcgcaggcgtatcgcttagccgaatgctggacaaagacccagagcttgagggccgtctagagaaaattttcgaagacacccgcgacgctgcctatcacattatcgacgccaagggctccacttcctacggcatcggcatgggtcttgctcgcatcacccgcgcaatcctgcagaaccaagacgttgcagtcccagtctctgcactgctccacggtgaatacggtgaggaagacatctacatcggcaccccagctgtggtgaaccgccgaggcatccgccgcgttgtcgaactagaaatcaccgaccacgagatggaacgcttcaagcattccgcaaataccctgcgcgaaattcagaagcagttcttctaaatctttggcgcctagttggcgacgcaagtgtttcattggaacacttgcgctgccaactttttggtttacgggcacaatgaaactgttggatggaatttagagtgtttgtagcttaaggagctcaaatgaatgagtttgaccaggacattctccaggagatcaagactgaactcgacgagttaattctagaacttgatgaggtgacacaaactcacagcgaggccatcgggcaggtctccccaacccattacgttggtgcccgcaacctcatgcattacgcgcatcttcgcaccaaagacctccgtggcctgcagcaacgcctctcctctgtgggagctacccgcttgactaccaccgaaccagcagtgcaggcccgcctcaaggccgcccgcaatgttatcggagctttcgcaggtgaaggcccactttatccaccctcagatgtcgtcgatgccttcgaagatgccgatgagattctcgacgagcacgccgaaattctccttggcgaacccctaccggatactccatcctgcatcatggtcaccctgcccaccgaagccgccaccgacattgaacttgtccgtggcttcgccaaaagcggcatgaatctagctcgcatcaactgtgcacacgacgatgaaaccgtctggaagcagatgatcgacaacgtccacaccgttgcagaagaagttggccgggaaatccgcgtcagcatggacctcgccggaccaaaagtacgcaccggcgaaatcgccccaggcgcagaagtaggtcgcgcacgagtaacccgcgacgaaaccggaaaagtactgacgcccgcaaaactgtggatcaccgcccacggctccgaaccagtcccagcccccgaaagcctgcccggtcgccccgctctgccgattgaagtcaccccagaatggttcgacaaactagaaatcggcagcgtcatcaacgtcccagacacccgcg

SEQ ID NO: 44 - protein, 318 aa, Corynebacterium glutamicum
VGSKMKETVGNKIVLIGAGDVGVAYAYALINQGMADHLAIIDIDEKKLEGNVMDLNHGVVWADSRTRVTKGTYADCEDAAMVVICAGAAQKPGETRLQLVDKNVKIMKSIVGDVMDSGFDGIFLVASNPVDILTYAVWKFSGLEWNRVIGSGTVLDSARFRYMLGELYEVAPSSVHAYIIGEHGDTELPVLSSATIAGVSLSRMLDKDPELEGRLEKIFEDTRDAAYHIIDAKGSTSYGIGMGLARITRAILQNQDVAVPVSALLHGEYGEEDIYIGTPAVVNRRGIRRVVELEITDHEMERFKHSANTLREIQKQFF

SEQ ID NO: 45 - DNA, 4200 bp, Corynebacterium glutamicum
  CDS (956)..(1942)
  CDS (1945)..(3135)
cctgctggacctgcaccgacaacggcaacacgcaaagggcgagacatataaagttcgattccttaaaggggttctaaaaaatgtggagtatgtgagcgggggttccacttgtagattcgactcctatcggggtgcgactgctaatggtgccctgctatcaaccctccatgatacgtggtaagtgcagactaataaaggccagtcggggagtattgggggctttgctggggtcagatttgtcacgctgcgcgctttcatagaccccattaatggggggtgaagagctgtaaagtaccgctaaaaactttgcaaagggtgcttcgcaacttgtaaccgctccgtattgttttctacggcaataagcatttgtgctgctcaaagcgtggaattgagatcggtttgaaaattacaaaataaaactttgcaaaccgggctgtacgcaaggcggacgaacgctaaactatgtaagaaatcacaacctcccctcattagtgccaggaggcacaagcctgaagtgtcatcaatgagaaggttcaggctgaaattagaaaggcgatgtatgtctgacacaccgacctcagctctgatcaccacggtcaaccgcagcttcgatggattcgatttggaagaagtagcagcagaccttggagttcggctcacctacctgcccgacgaagaactagaagtatccaaagttctcgcggcggacctcctcgctgaggggccagctctcatcatcggtgtaggaaacacgtttttcgacgcccaggtcgccgctgccctcggcgtcccagtgctactgctggtagacaagcaaggcaagcacgttgctcttgctcgcacccaggtaaacaatgccggcgcagttgttgcagcagcatttaccgctgaacaagagccaatgccggataagctgcgcaaggctgtgcgcaaccacagcaacctcgaaccagtcatgagcgccgaactctttgaaaactggctgctcaagcgcgcacgcgcagagcactcccacattgtgctgccagaaggtgacgacgaccgcatcttgatggctgcccaccagctgcttgatcaagacatctgtgacatcacgatcctgggcgatccagtaaagatcaaggagcgcgctaccgaacttggcctgcaccttaacactgcatacctggtcaatccgctgacagatcctcgcctggaggaattcgccgaacaattcgcggagctgcgcaagtcaaagagcgtcactatcgatgaagcccgcgaaatcatgaaggatatttcctacttcggcaccatgatggtccacaacggcgacgccgacggaatggtatccggtgcagcaaacaccaccgcacacaccattaagccaagcttccagatcatcaaaactgttccagaagcatccgtcgtttcttccatcttcctcatggtgctgcgcgggcgactgtgggcattcggcgactgtgctgttaacccgaacccaactgctgaacagcttggtgaaatcgccgttgtgtcagcaaaaactgcagcacaatttggcattgatcctcgcgtagccatcttgtcctactccactggcaactccggcggaggctcagatgtggatcgcgccatcgacgctcttgcagaagcacgccgacttaacccagaactatgcgtcgatggaccacttcagttcgacgccgccgtcgacccgggtgtggcgcgcaagaagatgccagactctgacgtcgctggccaggcaaatgtgtttatcttccctgacctggaagccggaaacatcggctacaaaactgcacaacgcaccggtcacgccctggcagttggtccgattctgcagggcctaaacaaaccagtcaacgacctttcccgtggcgcaacagtccctgacatcgtcaacacagtagccatcacagcaattcaggcaggaggacgcagctaatggcattggcacttgttttgaactccggttcatcttccatcaaattccagctggtcaaccccgaaaactctgccatcgacgagccatatgtttctggtcttgtggagcagattggtgagccaaacggccgcatcgtactcaaaatagagggtgaaaaatataccctagagacacccatcgcagatcactccgaaggcctaaacctggcgttcgatctcatggaccagcacaactgtggtccttcccaactggaaatcaccgcagttggacaccgcgtggtccacggcggaatcttgttctccgcaccggaacttatcactgatgaaatcgtggaaatgatccgcgatctcattccactcgcaccactgcacaaccctgcaaacgttgacggcattgatgttgctcgaaaaattctccccgatgtcccacacgtagctgtctttgacaccggtttcttccactcacttccaccagcagctgcgctgtatgccatcaacaaggatgtcgcagctgaacacggaatcaggcgctatggtttccacggcacctcccatgaatttgtgtccaagcgcgtggtggaaattctggaaaagcccaccgaagacatcaacaccatcaccttccacctgggcaacggcgcatccatggctgctgttcaaggtggccgtgcggtagatacttccatgggtatgacacctctcgcgggccttgtcatgggtacccgaagcggtgacattgatccaggtatcgtcttccacctttcccgcaccgctggcatgagcatcgatgagatcgataatctgctgaacaaaaagtcgggtgtaaagggactttccggtgttaatgatttccgtgaactgcgggaaatgatcgacaacaatgatcaagatgcctggtccgcgtacaacatttacatacaccaactccgccgctacctcggttcctacatggtggcactgggacgggtagacaccatcgtgttcaccgccggtgtcggtgaaaatgcccagtttgtccgtgaggatgccttggcaggtttggaaatgtacggaattgagatcgatccagagcgtaacgcattgccaaacgatggtcctcgattgatttccaccgatgcctccaaggtgaaggtgtttgttattccaactaatgaagagttagctatcgctaggtacgcggtgaagttcgcttagctctcctggttaggatccaccacaaatcgctctgatcagcggttttgtggtggatttttgcgtttttaaggggtgaaactgcacggatccaccacagatcccagttttcctttggaacgtggtggatccttgccctggagcttcacaggaatcgcttgttggcccctagacctcttggggttgcgaattttcgtccccaccgaacattaaaaggccggttttggtcgaaaatttgctctaacaccttgctattatgcgaatattcgttccatttcatcgaattccagcaacccgtaacgagaagttgaacaggaaacctgcagtaaccccgcagaaatcacatcagccccaattgtcccaaaagtaactcccccagaatcgcttctaagggcctaactcgcccaaagtcaaactaggggacatcgcactcctaaaggcccttaaatcgccacctaccaaatagccccaagtcaaaacagctagaaccaactcagtggccgcacggcattcgccatatccacaagtgcgtaacggtggtgcgggaacggtgcagaacgtgcctgaatgcggagtgcctcggagatgccggtgcgcaggcctttttgggagaacgggtattcaaacaaagggttcgcggaggcggaagctttgagttttcgctctcgaagccagctgaggcctgccgacatgatggcaattttgatttggttgaagcggggttcgtttgtggggatttcggtgagtcggcgggcagcccgtcggatgcgggattcactcaaattggagctgaccagcaacaagatggtggtgagggtggccattcggtagtgggtggatgattgggggagtttgtctagggcttggactgcgagttcgatttggttttcggccatgagttggcgggcgagcccgaacgcggaggacacggtggtggggttggttgcccagacaagtgcgtagaggcgcagtgaatggaagcggactacgtgtgggtcgctggagatgtgggaccaggtgtcgctgagtgattcgaaggcggaggcgtcgaggtcttcgaaatct

SEQ ID NO: 46 - protein, 329 aa, Corynebacterium glutamicum
MSAELFENWLLKRARAEHSHIVLPEGDDDRILMAAHQLLDQDICDITILGDPVKIKERATELGLHLNTAYLVNPLTDPRLEEFAEQFAELRKSKSVTIDEAREIMKDISYFGTMMVHNGDADGMVSGAANTTAHTIKPSFQIIKTVPEASVVSSIFLMVLRGRLWAFGDCAVNPNPTAEQLGEIAVVSAKTAAQFGIDPRVAILSYSTGNSGGGSDVDRAIDALAEARRLNPELCVDGPLQFDAAVDPGVARKKMPDSDVAGQANVFIFPDLEAGNIGYKTAQRTGHALAVGPILQGLNKPVNDLSRGATVPDIVNTVAITAIQAGGRS

SEQ ID NO: 47 - protein, 397 aa, Corynebacterium glutamicum
MALALVLNSGSSSIKFQLVNPENSAIDEPYVSGLVEQIGEPNGRIVLKIEGEKYTLETPIADHSEGLNLAFDLMDQHNCGPSQLEITAVGHRVVHGGILFSAPELITDEIVEMIRDLIPLAPLHNPANVDGIDVARKILPDVPHVAVFDTGFFHSLPPAAALYAINKDVAAEHGIRRYGFHGTSHEFVSKRVVEILEKPTEDINTITFHLGNGASMAAVQGGRAVDTSMGMTPLAGLVMGTRSGDIDPGIVFHLSRTAGMSIDEIDNLLNKKSGVKGLSGVNDFRELREMIDNNDQDAWSAYNIYIHQLRRYLGSYMVALGRVDTIVFTAGVGENAQFVREDALAGLEMYGIEIDPERNALPNDGPRLISTDASKVKVFVIPTNEELAIARYAVKFA

SEQ ID NO: 48 - DNA, 3780 bp, Corynebacterium glutamicum
  CDS (996)..(2732)
taatgaggaaaaccgaaccccaccagaagaattccaacagcgcaccaccaatgatcgggcctgccgcagcgccaagaattgccacggaaccccaaataccaattgcagtgttgcgctcacgctcatcctcaaacgtaatgcggatcagagccaaggttgcaggcatcatcgttgccgcaccgatgccaaggaaagctctcgcagcaacaagagcccacgcagttggagcaaacgcagcaccaagtgaagcgattccgaaaatgctcaagcccatgaggaacatccggcggtggccgattttgtcacccaaagtgccggtacccaaaagaaggcccgccatgagcaggggatatgcgttgatgatccacaacgcttgggtttcggtggctgcgagctgttcacgcagcagagggagtgcggtgtagagaatcgagttgtctacaccgatcagaaagagaccaccgctgataacggcgaggaaagcccaacgttgggttttcgtaggcgcttgcgcctgtaaggtttctgaagtcatggatcgtaactgtaacgaatggtcggtacagttacaactcttttgttggtgttttagaccacggcgctgtgtggcgatttaagacgtcggaaatcgtaggggactgtcagcgtgggtcgggttctttgaggcgcttagaggcgattctgtgaggtcactttttgtggggtcggggtctaaatttggccagttttcgaggcgaccagacaggcgtgcccacgatgtttaaataggcgatcggtgggcatctgtgtttggtttcgacgggctgaaaccaaaccagactgcccagcaacgacggaaatcccaaaagtgggcatccctgtttggtaccgagtacccacccgggcctgaaactccctggcaggcgggcgaagcgtggcaacaactggaatttaagagcacaattgaagtcgcaccaagttaggcaacacaatagccataacgttgaggagttcagatggcacacagctacgcagaacaattaattgacactttggaagctcaaggtgtgaagcgaatttatggtttggtgggtgacagccttaatccgatcgtggatgctgtccgccaatcagatattgagtgggtgcacgttcgaaatgaggaagcggcggcgtttgcagccggtgcggaatcgttgatcactggggagctggcagtatgtgctgcttcttgtggtcctggaaacacacacctgattcagggtctttatgattcgcatcgaaatggtgcgaaggtgttggccatcgctagccatattccgagtgcccagattggttcgacgttcttccaggaaacgcatccggagattttgtttaaggaatgctctggttactgcgagatggtgaatggtggtgagcagggtgaacgcattttgcatcacgcgattcagtccaccatggcgggtaaaggtgtgtcggtggtagtgattcctggtgatatcgctaaggaagacgcaggtgacggtacttattccaattccactatttcttctggcactcctgtggtgttcccggatcctactgaggctgcagcgctggtggaggcgattaacaacgctaagtctgtcactttgttctgcggtgcgggcgtgaagaatgctcgcgcgcaggtgttggagttggcggagaagattaaatcaccgatcgggcatgcgctgggtggtaagcagtacatccagcatgagaatccgtttgaggtcggcatgtctggcctgcttggttacggcgcctgcgtggatgcgtccaatgaggcggatctgctgattctattgggtacggatttcccttattctgatttccttcctaaagacaacgttgcccaggtggatatcaacggtgcgcacattggtcgacgtaccacggtgaagtatccggtgaccggtgatgttgctgcaacaatcgaaaatattttgcctcatgtgaaggaaaaaacagatcgttccttccttgatcggatgctcaaggcacacgagcgtaagttgagctcggtggtagagacgtacacacataacgtcgagaagcatgtgcctattcaccctgaatacgttgcctctattttgaacgagctggcggataaggatgcggtgtttactgtggataccggcatgtgcaatgtgtggcatgcgaggtacatcgagaatccggagggaacgcgcgactttgtgggttcattccgccacggcacgatggctaatgcgttgcctcatgcgattggtgcgcaaagtgttgatcgaaaccgccaggtgatcgcgatgtgtggcgatggtggtttgggcatgctgctgggtgagcttctgaccgttaagctgcaccaacttccgctgaaggctgtggtgtttaacaacagttctttgggcatggtgaagttggagatgctcgtggagggacagccagaatttggtactgaccatgaggaagtgaatttcgcagagattgcggcggctgcgggtatcaaatcggtacgcatcaccgatccgaagaaagttcgcgagcagctagctgaggcattggcatatcctggacctgtactgatcgatatcgtcacggatcctaatgcgctgtcgatcccaccaaccatcacgtgggaacaggtcatgggattcagcaaggcggccacccgaaccgtctttggtggaggagtaggagcgatgatcgatctggcccgttcgaacataaggaatattcctactccatgatgattgatacacctgctgttctcattgaccgcgagcgcttaactgccaacatttccaggatggcagctcacgccggtgcccatgagattgccctgcgtccgcatgtgaaaacgcacaaaatcattgaaattgcgcagatgcaggtcgacgccggtgcccgagggatcacctgcgcaaccattggcgaggcggaaatttttgccggcgcaggttttacggacatctttattgcatatccgctgtatctaaccgatcatgcagtgcaacgcctgaacgcgatccccggagaaatttccattggcgtggattcggtagagatggcacaggcgacggcgggtttgcgggaagatatcaaggctctgattgaagtggattcgggacatcgtagaagtggagtcacggcgactgcttcagaattgagtcagatccgcgaggcgctgggcagcaggtatgcaggagtgtttacttttcctgggcattcttatggcccgggaaatggtgagcaggcagcagctgatgagcttcaggctctaaacaacagcgtccagcgacttgctggcggcctgacttctggcggttcctcgccgtctgcgcagtttacagacgcaatcgatgagatgcgaccaggcgtgtatgtgtttaacgattcccagcagatcacctcgggagcatgcactgagaagcaggtggcaatgacggtgctgtctactgtggtcagccgaaatgtgtcagatcgtcggatcattttggatgcgggatccaaaatcctcagcactgataaaccagcatggattgatggcaatggttttgttctggggaatcctgaagcccgaatctctgctttgtcggagcatcacgcaaccattttctggccagataaagtgctacttccagtaatcggggagcagctcaacatcgtgcccaaccatgcctgcaacgtgattaatttggtggatgaggtctacgttcgggaagccgatggcactttccgtacctggaaggtagttgcccgcggcagaaacaattagggaaacctcttgacctt

SEQ ID NO: 49 - protein, 579 aa, Corynebacterium glutamicum
MAHSYAEQLIDTLEAQGVKRIYGLVGDSLNPIVDAVRQSDIEWVHVRNEEAAAFAAGAESLITGELAVCAASCGPGNTHLIQGLYDSHRNGAKVLAIASHIPSAQIGSTFFQETHPEILFKECSGYCEMVNGGEQGERILHHAIQSTMAGKGVSVVVIPGDIAKEDAGDGTYSNSTISSGTPVVFPDPTEAAALVEAINNAKSVTLFCGAGVKNARAQVLELAEKIKSPIGHALGGKQYIQHENPFEVGMSGLLGYGACVDASNEADLLILLGTDFPYSDFLPKDNVAQVDINGAHIGRRTTVKYPVTGDVAATIENILPHVKEKTDRSFLDRMLKAHERKLSSVVETYTHNVEKHVPIHPEYVASILNELADKDAVFTVDTGMCNVWHARYIENPEGTRDFVGSFRHGTMANALPHAIGAQSVDRNRQVIAMCGDGGLGMLLGELLTVKLHQLPLKAVVFNNSSLGMVKLEMLVEGQPEFGTDHEEVNFAEIAAAAGIKSVRITDPKKVREQLAEALAYPGPVLIDIVTDPNALSIPPTITWEQVMGFSKAATRTVFGGGVGAMIDLARSNIRNIPTP

SEQ ID NO: 50 - DNA, 3600 bp, Corynebacterium glutamicum
  CDS (1037)..(2542)
gaagcgctacggacttcgcgccggcgtcgacagcaatgcgtccagcatccaagtgagtatggtgctcatcatcaataccaacgcggaacttcaccgtcaccggaatgtccgtgccttccgtagccttcacagccgcggaaacgatgttttcaaacaaacggcgcttgtaaggaatcgcagaaccgccaccccggcgcgtgacctttggaaccgggcagccaaagttcatatcaatatgatccgccaagttttcatcaacgatcatcttcgccgcttcgtaggtgtacttcgggtcaaccgtgtacagctgcaagcttcggggattttcatccggcgcgaaggtggtcatgtgcatggttttctcattgcgctcaacaagagcacgcgcagtcaccatttcacagacgtacagccccgagattgttcccgtgcgttgcatttcctgttcacggcacagcgtgcggaaagcaacgttggttacaccagccatgggggctagaaccacaggggaggcaaggtcaaaggggccgatttttaaagtcacctaactattgtcccccgtgaatcaggttgggcaaaatatttgaagcaaattgtgagcagggcgcaactaggaaagtggtgtgctttcactttttgggggctggggttgggttaagcttcgcgggctctagggttggtctgagctttattcctgggctttgggaggcttgcaaacagggggcatgcaaatttgggggtaatgctgggccttgaaatcccactatcacagatagtattcgggcatttcctgtcacgatggtttatccttgggacacaacatcaaagtggggtacatcatatgcttccggttgaagtgacctatctgaaaagattggtcgaaccttgaagcaatggtgtgaactgcgttaacgaattttgtcggacgttaaaatggtcgcattctgcttgctgaagtggcacacctatgtgttctgcttgggtatagcagtgcgggaaaaatttgaaaaagtccgattacctgaggaggtattcaatgtctgatcgcattgcttcagaaaagctgcgctccaagctcatgtccgccgacgaggcggcacagtttgttaaccacggtgacaaggttggtttctccggcttcaccggcgctggctacccaaaggcactgcctacggcaatcgctaaccgggctaaagaagcacacggtgcaggcaacgactacgcaatcgacctgttcactggcgcatcgaccgcccctgactgcgatggcgtacttgcagaagctgacgctatccgctggcgcatgccatacgcatctgatccaatcatgcgtaacaagatcaactccggctccatgggatactccgatatccacctgtcccactccggccagcaggttgaagagggcttcttcggccagctcaacgtagctgtcattgaaatcacccgcatcactgaagagggctacatcatcccttcttcctccgtgggtaacaacgttgagtggctcaacgctgcagagaaggtcatcctcgaggttaactcttggcagtctgaagacctcgaaggtatgcacgacatctggtctgttcctgccctgccaaaccgcattgccgtgccaatcaacaagccaggcgaccgcatcggtaagacctacatcgagttcgacaccgacaaggttgttgctgttgttgagaccaacaccgcagaccgcaacgcaccattcaagcctgtcgacgacatctctaagaagatcgctggcaacttcctcgacttcctggaaagcgaagttgctgcaggtcgcctgtcctacgacggctacatcatgcagtccggcgtgggcaacgtgccaaacgcggtgatggcaggcctgctggaatccaagtttgagaacatccaggcctacaccgaagttatccaggacggcatggtggacctcatcgacgccggcaagatgaccgttgcatccgcaacttccttctccctgtctcctgagtacgcagagaagatgaacaacgaggctaagcgttaccgcgagtccattatcctgcgcccacagcagatctctaaccacccagaggtcatccgccgcgttggcctgatcgccaccaacggtctcatcgaggctgacatttacggcaacgtcaactccaccaacgtttctggctcccgcgtcatgaacggcatcggcggctccggcgacttcacccgtaacggctacatctccagcttcatcaccccttcagaggcaaagggcggcgcaatctctgcgatcgttcctttcgcatcccacatcgaccacaccgagcacgatgtcatggttgttatctctgagtacggttacgcagaccttcgtggtctggctccacgtgagcgcgttgccaagatgatcggcctggctcaccctgattaccgcccactgctcgaggagtactacgctcgcgcaacctccggtgacaacaagtacatgcagacccctcatgatcttgcaaccgcgtttgatttccacatcaacctggctaagaacggctccatgaaggcataagttttttcttggtttagaaaccgccgcctcgacaacatttcgaggcggcggtttcttttattacctgggttttgagcgttaaattagaccaggtcaggctagtgtttggtagctaattgagggcgattttaataaggccggtgccatgtactaatatggtctgagttgggcctatagctcagttggtagagctacggacttttaatccgcaggtcttgggttcgagtcccaatgggcccacatcttaagtacccctgttttggagaatgctccgagccaggggtacttttcttttcctcacacacagtagctgctgagaaaaatgaagaccttttgttaggttgggagtatgaccaacccatacgaggccttcataccgctcaagcatcgtacggggattgaacccgagcacaccttttgggaatgggaaaacaaaagggttcacattgcaaggagacgtcgagaagcgcccgtccgcgttatcgtggtgcatgggctaggcacccatagtggcgccctctggcccctcgtcgcggccattgagggcgcggacctcgccgcgatcgacctgcctaaaactccgctttacgacgattggctgcgccttttagaatctttcatctcttccgaagacgacggtcggccactcatcctgatcggtgcaggcaccggaggcttgctttgcgcagaagctgcacaccgcacaggactggtcgcacacgtcattgccacctgcctgctcaacccctccgaccagccgacgcgccgggcactgttcaggttttcaccgctgactcggttgatccaaggccgcttgcgcaaccgcgaaattcccgtgaccagagtgttgaacttcagcaaaatcagccgcagcccagccctgagcaaattgtgcgcggccgatgaatttagcggagcatccaaaataacctggggtttcctcgcgtcatatgtgcaacacaaggccaaactgggtgcagttcccgtcactctgatgcaccctgaccacgaccttctgactcccgttgagctcagtctgcgtacgctttcgcgcc

SEQ ID NO: 51 - protein, 502 aa, Corynebacterium glutamicum
MSDRIASEKLRSKLMSADEAAQFVNHGDKVGFSGFTGAGYPKALPTAIANRAKEAHGAGNDYAIDLFTGASTAPDCDGVLAEADAIRWRMPYASDPIMRNKINSGSMGYSDIHLSHSGQQVEEGFFGQLNVAVIEITRITEEGYIIPSSSVGNNVEWLNAAEKVILEVNSWQSEDLEGMHDIWSVPALPNRIAVPINKPGDRIGKTYIEFDTDKVVAVVETNTADRNAPFKPVDDISKKIAGNFLDFLESEVAAGRLSYDGYIMQSGVGNVPNAVMAGLLESKFENIQAYTEVIQDGMVDLIDAGKMTVASATSFSLSPEYAEKMNNEAKRYRESIILRPQQISNHPEVIRRVGLIATNGLIEADIYGNVNSTNVSGSRVMNGIGGSGDFTRNGYISSFITPSEAKGGAISAIVPFASHIDHTEHDVMVVISEYGYADLRGLAPRERVAKMIGLAHPDYRPLLEEYYARATSGDNKYMQTPHDLATAFDFHINLAKNGSMKA

SEQ ID NO: 52 - DNA, 2100 bp, Corynebacterium glutamicum
  CDS (850)..(1131)
ggccgattccgaaggaaatcgcacgcctggattttgataacggtgatccaaacgcagcagctcctgttccaggtttgagcccatttatgcccaaagagcaggcaattgagcggcttttagagatcattggacagaacctgctgctgccttttgagatcgaggtgccggagaaaattcagcgggaagcactgagggatttcacggcggaaactcaattgggttccaccgtgggcgctgatatttttgatgcattgcaaatggctgttggggtggtgtcgggaagcgcgaagagcaattggcgcaaatggggagcgtttggtgtgggggctgcagctttgaccgctgcaacgggtggtttggctttggcggctgtgccgactgttgctggagtagccactgttgcctcgacactcgcagcatttggtccaggtgggatgatgggcggtttggtcactgcaggaacactgctcacagttggtggcggcagtttaaccgctggggtgttgagctcggtgaacaccacggaagagatcgaagcgctcgttgtacagaagctaagtttggctattttgtggcagcgccatgagatagatagaactcatgaggtgtgggaagaattcgcggaggcagaacgtctgattgtgcgggagcacacgcgtgtgaaaaacgtgtcggatagttcttcgcccattttgaaagctttcgagcagcagcgttcgactattgagcgggcgttgaagtatttgagcgatcatgggatggaacctggctggtttgaagaactcgaaccaccagccccaacaccgtttctaaaactgcgggctaagaaaactgattaggagaaacacaatggagaaagttcgtctgactgcttttgttcatggtcatgtccagggcgtgggttttcgatggtggactacctcgcaggcacgagaattaaaacttgcaggttctgccagtaatttaagtgacggccgggtgtgcgtggttgctgaagggccacaaacacagtgcgaagaactgctgagaaggttgaaggaaaaccccagctcgtatcgcagaccaggtcatgtggacacagttattgagcaatggggcgagccgcgtgacgttgaaggctttgtggagcgctagactttaaccccgttatgtatttgaaatcgttgacgctcaaggggtttaagtctttcgcgtctgcgacgaccctgaaatttgagccaggcatttgtgccgtggtgggtccgaatggttcaggcaaatccaatgtggttgatgcgctggcctgggtgatgggtgaaggttctgcgaagaccttgcgtggcggcaaaatggaagatgtcatttttgctggcgcgggcgatcgtaaaccgttgggtcgcgcagaagtcacgctgaccattgataactctgatggcgcactgcccattgagtacaccgaagtgtcggtgaccagacggatgttccgtgatggtgcaagtgaatatgagatcaatggggcgaaagctcgattgatggatatccaggagctgttgtcggataccggtattggccgtgaaatgcacatcatggtggggcagggaaagctcgcagagattttggagtcccgccccgaagagcgccgagcgtatatcgaagaagctgcgggtgtgctcaagcaccggcgcaggaaagaaaaggcgcagcgcaaacttcagggcatgcaggtcaatcttgatcgtttgcaggatctgacccatgagttggccaagcagctcaagccgttggctaggcaggcggaagcagcgcagcgtgcggcgacggtgcaggctgatttgcgtgatgcgcgtttccagattgctggctttgagatcgtgaagctctcggaaaagctggaaacctctactgagcgcgagaaaatgattcgtgagcaggcggaagcagcacaagagcagctggaagaagccaccacaactcagatggaagtggagatggagttggcggagatcactccgcaggctgaagctgcgcaacagttgtggtttgatttgtcttcgctggctgagcgggtgtcggcaacgatgcgtattgctgcagaccgtgcgagttcaggtgccgcggatg

SEQ ID NO: 53 - protein, 94 aa, Corynebacterium glutamicum
MEKVRLTAFVHGHVQGVGFRWWTTSQARELKLAGSASNLSDGRVCVVAEGPQTQCEELLRRLKENPSSYRRPGHVDTVIEQWGEPRDVEGFVER

SEQ ID NO: 54 - DNA, 24 bp, Artificial Sequence
  Description of Artificial Sequence Synthetic primer
cagcttcttcgatatacgctcgcc

SEQ ID NO: 55 - DNA, 46 bp, Artificial Sequence
  Description of Artificial Sequence Synthetic primer
ctgattaggagaaacacaatggaggtggagcgctagactttaaccc

SEQ ID NO: 56 - DNA, 46 bp, Artificial Sequence
  Description of Artificial Sequence Synthetic primer
gggttaaagtctagcgctccacctccattgtgtttctcctaatcag

SEQ ID NO: 57 - DNA, 25 bp, Artificial Sequence
  Description of Artificial Sequence Synthetic primer
gctgcagctttgaccgctgcaacgg

SEQ ID NO: 58 - DNA, 33 bp, Artificial Sequence
  Description of Artificial Sequence Synthetic primer
gggaatctagacccaccatgatgtgcatttcac

SEQ ID NO: 59 - DNA, 34 bp, Artificial Sequence
  Description of Artificial Sequence Synthetic primer
gggaatctagagttgctggagtagccactgttgc

SEQ ID NO: 60 - DNA, 3423 bp, Corynebacterium glutamicum
  CDS (1)..(3420)
gtgtcgactcacacatcttcaacgcttccagcattcaaaaagatcttggtagcaaaccgcggcgaaatcgcggtccgtgctttccgtgcagcactcgaaaccggtgcagccacggtagctatttacccccgtgaagatcggggatcattccaccgctcttttgcttctgaagctgtccgcattggtaccgaaggctcaccagtcaaggcgtacctggacatcgatgaaattatcggtgcagctaaaaaagttaaagcagatgccatttacccgggatacggcttcctgtctgaaaatgcccagcttgcccgcgagtgtgcggaaaacggcattacttttattggcccaaccccagaggttcttgatctcaccggtgataagtctcgcgcggtaaccgccgcgaagaaggctggtctgccagttttggcggaatccaccccgagcaaaaacatcgatgagatcgttaaaagcgctgaaggccagacttaccccatctttgtgaaggcagttgccggtggtggcggacgcggtatgcgttttgttgcttcacctgatgagcttcgcaaattagcaacagaagcatctcgtgaagctgaagcggctttcggcgatggcgcggtatatgtcgaacgtgctgtgattaaccctcagcatattgaagtgcagatccttggcgatcacactggagaagttgtacacctttatgaacgtgactgctcactgcagcgtcgtcaccaaaaagttgtcgaaattgcgccagcacagcatttggatccagaactgcgtgatcgcatttgtgcggatgcagtaaagttctgccgctccattggttaccagggcgcgggaaccgtggaattcttggtcgatgaaaagggcaaccacgtcttcatcgaaatgaacccacgtatccaggttgagcacaccgtgactgaagaagtcaccgaggtggacctggtgaaggcgcagatgcgcttggctgctggtgcaaccttgaaggaattgggtctgacccaagataagatcaagacccacggtgcagcactgcagtgccgcatcaccacggaagatccaaacaacggcttccgcccagataccggaactatcaccgcgtaccgctcaccaggcggagctggcgttcgtcttgacggtgcagctcagctcggtggcgaaatcaccgcacactttgactccatgctggtgaaaatgacctgccgtggttccgactttgaaactgctgttgctcgtgcacagcgcgcgttggctgagttcaccgtgtctggtgttgcaaccaacattggtttcttgcgtgcgttgctgcgggaagaggacttcacttccaagcgcatcgccaccggattcattgccgatcacccgcacctccttcaggctccacctgctgatgatgagcagggacgcatcctggattacttggcagatgtcaccgtgaacaagcctcatggtgtgcgtccaaaggatgttgcagctcctatcgataagctgcctaacatcaaggatctgccactgccacgcggttcccgtgaccgcctgaagcagcttggcccagccgcgtttgctcgtgatctccgtgagcaggacgcactggcagttactgataccaccttccgcgatgcacaccagtctttgcttgcgacccgagtccgctcattcgcactgaagcctgcggcagaggccgtcgcaaagctgactcctgagcttttgtccgtggaggcctggggcggcgcgacctacgatgtggcgatgcgtttcctctttgaggatccgtgggacaggctcgacgagctgcgcgaggcgatgccgaatgtaaacattcagatgctgcttcgcggccgcaacaccgtgggatacaccccgtacccagactccgtctgccgcgcgtttgttaaggaagctgccagctccggcgtggacatcttccgcatcttcgacgcgcttaacgacgtctcccagatgcgtccagcaatcgacgcagtcctggagaccaacaccgcggtagccgaggtggctatggcttattctggtgatctctctgatccaaatgaaaagctctacaccctggattactacctaaagatggcagaggagatcgtcaagtctggcgctcacatcttggccattaaggatatggctggtctgcttcgcccagctgcggtaaccaagctggtcaccgcactgcgccgtgaattcgatctgccagtgcacgtgcacacccacgacactgcgggtggccagctggcaacctactttgctgcagctcaagctggtgcagatgctgttgacggtgcttccgcaccactgtctggcaccacctcccagccatccctgtctgccattgttgctgcattcgcgcacacccgtcgcgataccggtttgagcctcgaggctgtttctgacctcgagccgtactgggaagcagtgcgcggactgtacctgccatttgagtctggaaccccaggcccaaccggtcgcgtctaccgccacgaaatcccaggcggacagttgtccaacctgcgtgcacaggccaccgcactgggccttgcggatcgtttcgaactcatcgaagacaactacgcagccgttaatgagatgctgggacgcccaaccaaggtcaccccatcctccaaggttgttggcgacctcgcactccacctcgttggtgcgggtgtggatccagcagactttgctgccgatccacaaaagtacgacatcccagactctgtcatcgcgttcctgcgcggcgagcttggtaaccctccaggtggctggccagagccactgcgcacccgcgcactggaaggccgctccgaaggcaaggcacctctgacggaagttcctgaggaagagcaggcgcacctcgacgctgatgattccaaggaacgtcgcaatagcctcaaccgcctgctgttcccgaagccaaccgaagagttcctcgagcaccgtcgccgcttcggcaacacctctgcgctggatgatcgtgaattcttctacggcctggtcgaaggccgcgagactttgatccgcctgccagatgtgcgcaccccactgcttgttcgcctggatgcgatctctgagccagacgataagggtatgcgcaatgttgtggccaacgtcaacggccagatccgcccaatgcgtgtgcgtgaccgctccgttgagtctgtcaccgcaaccgcagaaaaggcagattcctccaacaagggccatgttgctgcaccattcgctggtgttgtcaccgtgactgttgctgaaggtgatgaggtcaaggctggagatgcagtcgcaatcatcgaggctatgaagatggaagcaacaatcactgcttctgttgacggcaaaatcgatcgcgttgtggttcctgctgcaacgaaggtggaaggtggcgacttgatcgtcgtcgtttcctaa

SEQ ID NO: 61 - protein, 1140 aa, Corynebacterium glutamicum
VSTHTSSTLPAFKKILVANRGEIAVRAFRAALETGAATVAIYPREDRGSFHRSFASEAVRIGTEGSPVKAYLDIDEIIGAAKKVKADAIYPGYGFLSENAQLARECAENGITFIGPTPEVLDLTGDKSRAVTAAKKAGLPVLAESTPSKNIDEIVKSAEGQTYPIFVKAVAGGGGRGMRFVASPDELRKLATEASREAEAAFGDGAVYVERAVINPQHIEVQILGDHTGEVVHLYERDCSLQRRHQKVVEIAPAQHLDPELRDRICADAVKFCRSIGYQGAGTVEFLVDEKGNHVFIEMNPRIQVEHTVTEEVTEVDLVKAQMRLAAGATLKELGLTQDKIKTHGAALQCRITTEDPNNGFRPDTGTITAYRSPGGAGVRLDGAAQLGGEITAHFDSMLVKMTCRGSDFETAVARAQRALAEFTVSGVATNIGFLRALLREEDFTSKRIATGFIADHPHLLQAPPADDEQGRILDYLADVTVNKPHGVRPKDVAAPIDKLPNIKDLPLPRGSRDRLKQLGPAAFARDLREQDALAVTDTTFRDAHQSLLATRVRSFALKPAAEAVAKLTPELLSVEAWGGATYDVAMRFLFEDPWDRLDELREAMPNVNIQMLLRGRNTVGYTPYPDSVCRAFVKEAASSGVDIFRIFDALNDVSQMRPAIDAVLETNTAVAEVAMAYSGDLSDPNEKLYTLDYYLKMAEEIVKSGAHILAIKDMAGLLRPAAVTKLVTALRREFDLPVHVHTHDTAGGQLATYFAAAQAGADAVDGASAPLSGTTSQPSLSAIVAAFAHTRRDTGLSLEAVSDLEPYWEAVRGLYLPFESGTPGPTGRVYRHEIPGGQLSNLRAQATALGLADRFELIEDNYAAVNEMLGRPTKVTPSSKVVGDLALHLVGAGVDPADFAADPQKYDIPDSVIAFLRGELGNPPGGWPEPLRTRALEGRSEGKAPLTEVPEEEQAHLDADDSKERRNSLNRLLFPKPTEEFLEHRRRFGNTSALDDREFFYGLVEGRETLIRLPDVRTPLLVRLDAISEPDDKGMRNVVANVNGQIRPMRVRDRSVESVTATAEKADSSNKGHVAAPFAGVVTVTVAEGDEVKAGDAVAIIEAMKMEATITASVDGKIDRVVVPAATKVEGGDLIVVVS